SEQUENCE LISTING (30 sequences)

SEQ ID NO: 1 - DNA, 4524 bp, Arabidopsis thaliana
  CDS (1)...(4524): Atino80 sequence
atggatccttcaagacgaccaccgaaggactctccttacgcgaatctattcgatctcgagccgttgatgaagtttagaattccgaaacctgaagatgaagttgattattatgggagtagtagccaggatgaaagtagaagcactcaaggtggggtagtggcaaactacagcaatgggtctaaatcgagaatgaatgcgagctccaagaagagaaagcggtggacagaagctgaggatgcagaggacgatgatgatctctacaatcaacatgttactgaggagcactaccgatcaatgcttggggagcatgtacaaaaattcaaaaataggtccaaggagactcaagggaatcctcctcatctgatgggttttccggtgctaaagagcaatgtgggcagttacagaggtaggaaaccagggaatgattaccatgggaggttctatgacatggacaactctccaaattttgcagctgatgtgaccccacataggcgaggaagctaccatgatcgtgatattacacccaagatagcatatgaaccttcgtatttggacattggtgatggtgtcatctacaaaatccccccaagttatgacaagctggtggcatcattaaacttaccgagcttttcagacattcatgtggaagaattttacttgaaaggaactctggatctgagatcattagcagaactgatggcaagtgataaaaggtctggagtaagaagccgtaatggaatgggtgagcctcgacctcaatatgaatctcttcaagctagaatgaaggccctgtcaccttcaaactccaccccaaattttagcctcaaggtgtcagaagctgcaatgaattctgccattccagaaggatctgctggaagtactgcacggacaattctgtctgagggtggtgttttacaggtccattacgtgaagattctggagaagggggatacatacgagattgttaaacgaagtctaccgaagaagctgaaagcaaagaatgatcctgcagtcattgagaaaacagaaagggataaaattagaaaagcctggatcaatattgtcagaagagatatagcaaaacaccatagaattttcactacttttcatcgtaaactatcaattgatgccaagaggtttgcagatggttgccaaagagaggtgagaatgaaggtgggtagatcatacaaaatcccaagaactgcaccaattcgcactaggaagatatccagagacatgctgctattctggaagcgatatgacaagcagatggcagaagagaggaaaaagcaagaaaaggaagctgcagaggcttttaaacgtgaacaggagcagcgagagtcaaaaaggcagcaacaaaggctcaatttccttattaaacagactgagctttacagtcacttcatgcaaaacaagaccgattcgaatccttccgaagccttaccaataggtgatgaaaatccgattgacgaagtgctcccagaaacttcagcggcagaaccttctgaggtagaggatcctgaagaggctgaactgaaggaaaaggtcttgagagctgcccaagatgcggtgtctaagcagaagcaaataacagatgcatttgacactgaatatatgaagctacgccaaacttctgaaatggaaggtcctttaaatgatatatcagtttctggctcgagcaatatagatttgcataacccatctacaatgcctgttacatcaacagttcagactccagagttatttaaaggaacccttaaagaataccaaatgaaaggccttcagtggctagtcaattgttatgagcagggtttgaatggcatacttgctgatgaaatgggcttgggtaagactattcaagctatggcgttcttggcacatttggctgaggaaaagaacatttggggtccatttcttgttgttgcccctgcctctgttcttaacaattgggctgatgaaatcagtcgtttctgtcctgacttgaaaactcttccatattggggaggattacaagaacgaacaattttaagaaagaatatcaatcccaagcgtatgtaccgaagggatgctggctttcatattttgattactagctatcagctattagtcactgatgaaaagtattttcgccgggtgaagtggcaatatatggtgctagatgaggcccaagcaatcaagagttcctccagtataagatggaaaacccttcttagttttaactgtcggaaccgattgcttctgactggtactccaattcagaacaacatggcagagttatgggccctgctgcatttcatcatgccaatgttgtttgacaaccatgatcaatttaatgaatggttctcaaaaggaattgagaatcatgctgaacacggaggcactttaaatgagcaccagcttaacagactgcatgcgatcttgaaaccgttcatgcttcgacgggtaaaaaaggatgtggtttctgagctaactacaaagacggaagttacagtacactgcaagctcagttctcgacaacaagctttttatcaggctattaagaacaaaatttctctggctgagttgtttgatagcaaccgcggacaatttactgataagaaagtattgaatttaatgaatattgtcattcaactaaggaaggtttgcaaccatccagagttgttcgaaaggaatgaagggagctcgtatctctactttggagtgacttccaattctcttttgccccatccctttggtgagctagaggatgtacattattctggtggtcaaaatccgataatatacaagatacctaagctactacaccaagaggtgctccaaaattctgaaacattttgttcttctgtcgggcgtggcatctcaagagaatcttttctgaagcattttaatatatattcacctgagtatattcttaagtcaatattcccatctgatagtggggtagatcaagtggttagtggaagtggagcatttggcttttcacgcttgatggatctatcaccatcagaagttggatatctggctctgtgttctgttgcagaaaggctattattttctatactgaggtgggagcggcaatttttggatgaattagttaactctcttatggagtccaaggatggtgatcttagtgacaataacatcgagagagttaaaaccaaagctgtcacaagaatgttgctgatgccatcaaaagttgaaacgaattttcagaaaaggagactaagcacagggcctacccgtccttcatttgaagcgctagtgatctctcatcaggataggtttctttcaagtatcaaactcctgcattctgcatatacttatatcccaaaagccagagctccacctgtaagcattcattgctcggacagaaattcggcatacagagttacagaagaattacatcaaccatggcttaagagactattaatcggttttgcacgaacgtcagaagctaatggacccaggaagcctaacagctttccacatcctttaatccaagaaattgattcagaacttccagttgtgcagcctgcgcttcaactgacacacagaatatttggttcttgccctccaatgcaaagttttgacccagcaaagttgctcacggactctgggaagctgcagacacttgatatattattgaagcggcttcgagctggaaatcacagggtgctcctgtttgcacaaatgacaaagatgctgaacattctcgaggattatatgaactatagaaagtacaagtacctcaggcttgatggatcctccaccatcatggatcgccgagatatggttagggattttcagcataggagcgatatttttgtattcttgctgagcaccagagctggaggacttggtatcaacttgacggctgcagacactgtcattttctatgaaagtgattggaatcccaccttggatttacaagctatggacagggctcatcgtcttggacagacaaaagatgttactgtttatcgtctcatctgtaaggagacggtggaagagaaaattttgcacagggcaagtcagaaaaatacagttcaacagcttgttatgactggagggcatgttcagggtgatgattttcttggagctgcggatgtggtatctctgctaatggatgatgcggaggcagcacaactggagcagaaattcagagaactaccattacaggtaaaggacaggcagaagaaaaagacgaaacgtatcagaatagatgctgaaggagatgcaactttggaagagttagaagatgttgaccgacaggataacggacaggaacctttggaagaaccggaaaagccaaaatccagtaataaaaagaggagagctgcttcaaatccgaaagctagagctcctcagaaagcaaaggaagaagcaaatggtgaagatactcctcagaggacaaaaagggtaaagagacaaacaaagagcataaacgaaagtcttgaacctgtattctctgcctctgtaacagaatcaaataaaggattcgatccaagtagctccgctaactaa

SEQ ID NO: 2 - protein, 1507 aa, Arabidopsis thaliana
MDPSRRPPKDSPYANLFDLEPLMKFRIPKPEDEVDYYGSSSQDESRSTQGGVVANYSNGSKSRMNASSKKRKRWTEAEDAEDDDDLYNQHVTEEHYRSMLGEHVQKFKNRSKETQGNPPHLMGFPVLKSNVGSYRGRKPGNDYHGRFYDMDNSPNFAADVTPHRRGSYHDRDITPKIAYEPSYLDIGDGVIYKIPPSYDKLVASLNLPSFSDIHVEEFYLKGTLDLRSLAELMASDKRSGVRSRNGMGEPRPQYESLQARMKALSPSNSTPNFSLKVSEAAMNSAIPEGSAGSTARTILSEGGVLQVHYVKILEKGDTYEIVKRSLPKKLKAKNDPAVIEKTERDKIRKAWINIVRRDIAKHHRIFTTFHRKLSIDAKRFADGCQREVRMKVGRSYKIPRTAPIRTRKISRDMLLFWKRYDKQMAEERKKQEKEAAEAFKREQEQRESKRQQQRLNFLIKQTELYSHFMQNKTDSNPSEALPIGDENPIDEVLPETSAAEPSEVEDPEEAELKEKVLRAAQDAVSKQKQITDAFDTEYMKLRQTSEMEGPLNDISVSGSSNIDLHNPSTMPVTSTVQTPELFKGTLKEYQMKGLQWLVNCYEQGLNGILADEMGLGKTIQAMAFLAHLAEEKNIWGPFLVVAPASVLNNWADEISRFCPDLKTLPYWGGLQERTILRKNINPKRMYRRDAGFHILITSYQLLVTDEKYFRRVKWQYMVLDEAQAIKSSSSIRWKTLLSFNCRNRLLLTGTPIQNNMAELWALLHFIMPMLFDNHDQFNEWFSKGIENHAEHGGTLNEHQLNRLHAILKPFMLRRVKKDVVSELTTKTEVTVHCKLSSRQQAFYQAIKNKISLAELFDSNRGQFTDKKVLNLMNIVIQLRKVCNHPELFERNEGSSYLYFGVTSNSLLPHPFGELEDVHYSGGQNPIIYKIPKLLHQEVLQNSETFCSSVGRGISRESFLKHFNIYSPEYILKSIFPSDSGVDQVVSGSGAFGFSRLMDLSPSEVGYLALCSVAERLLFSILRWERQFLDELVNSLMESKDGDLSDNNIERVKTKAVTRMLLMPSKVETNFQKRRLSTGPTRPSFEALVISHQDRFLSSIKLLHSAYTYIPKARAPPVSIHCSDRNSAYRVTEELHQPWLKRLLIGFARTSEANGPRKPNSFPHPLIQEIDSELPVVQPALQLTHRIFGSCPPMQSFDPAKLLTDSGKLQTLDILLKRLRAGNHRVLLFAQMTKMLNILEDYMNYRKYKYLRLDGSSTIMDRRDMVRDFQHRSDIFVFLLSTRAGGLGINLTAADTVIFYESDWNPTLDLQAMDRAHRLGQTKDVTVYRLICKETVEEKILHRASQKNTVQQLVMTGGHVQGDDFLGAADVVSLLMDDAEAAQLEQKFRELPLQVKDRQKKKTKRIRIDAEGDATLEELEDVDRQDNGQEPLEEPEKPKSSNKKRRAASNPKARAPQKAKEEANGEDTPQRTKRVKRQTKSINESLEPVFSASVTESNKGFDPSSSAN

SEQ ID NO: 3 - DNA, 4492 bp, Arabidopsis thaliana
  gene (1)...(4492): At3g57300
atggatccttcaagacgaccaccgaaggactctccttacgcgaatctattcgatctcgagccgttgatgaagtttagaattccgaaacctgaagatgaagttgattattatgggagtagtagccaggatgaaagtagaagcactcaaggtggggtagtggcaaactacagcaatgggtctaaatcgagaatgaatgcgagctccaagaagagaaagcggtggacagaagctgaggatgcagaggacgatgatgatctctacaatcaacatgttactgaggagcactaccgatcaatgcttggggagcatgtacaaaaattcaaaaataggtccaaggagactcaagggaatcctcctcatctgatgggttttccggtgctaaagagcaatgtgggcagttacagaggtaggaaaccagggaatgattaccatgggaggttctatgacatggacaactctccaaattttgcagctgatgtgaccccacataggcgaggaagctaccatgatcgtgatattacacccaagatagcatatgaaccttcgtatttggacattggtgatggtgtcatctacaaaatccccccaagttatgacaagctggtggcatcattaaacttaccgagcttttcagacattcatgtggaagaattttacttgaaaggaactctggatctgagatcattagcagaactgatggcaagtgataaaaggtctggagtaagaagccgtaatggaatgggtgagcctcgacctcaatatgaatctcttcaagctagaatgaaggccctgtcaccttcaaactccaccccaaattttagcctcaaggtgtcagaagctgcaatgaattctgccattccagaaggatctgctggaagtactgcacggacaattctgtctgagggtggtgttttacaggtccattacgtgaagattctggagaagggggatacatacgagattgttaaacgaagtctaccgaagaagctgaaagcaaagaatgatcctgcagtcattgagaaaacagaaagggataaaattagaaaagcctggatcaatattgtcagaagagatatagcaaaacaccatagaattttcactacttttcatcgtaaactatcaattgatgccaagaggtttgcagatggttgccaaagagaggtgagaatgaaggtgggtagatcatacaaaatcccaagaactgcaccaattcgcactaggaagatatccagagacatgctgctattctggaagcgatatgacaagcagatggcagaagagaggaaaaagcaagaaaaggaagctgcagaggcttttaaacgtgaacaggagcagcgagagtcaaaaaggcagcaacaaaggctcaatttccttattaaacagactgagctttacagtcacttcatgcaaaacaagaccgattcgaatccttccgaagccttaccaataggtgatgaaaatccgattgacgaagtgctcccagaaacttcagcggcagaaccttctgaggtagaggatcctgaagaggctgaactgaaggaaaaggtcttgagagctgcccaagatgcggtgtctaagcagaagcaaataacagatgcatttgacactgaatatatgaagctacgccaaacttctgaaatggaaggtcctttaaatgatatatcagtttctggctcgagcaatatagatttgcataacccatctacaatgcctgttacatcaacagttcagactccagagttatttaaaggaacccttaaagaataccaaatgaaaggccttcagtggctagtcaattgttatgagcagggtttgaatggcatacttgctgatgaaatgggcttgggtaagactattcaagctatggcgttcttggcacatttggctgaggaaaagaacatttggggtccatttcttgttgttgcccctgcctctgttcttaacaattgggctgatgaaatcagtcgtttctgtcctgacttgaaaactcttccatattggggaggattacaagaacgaacaattttaagaaagaatatcaatcccaagcgtatgtaccgaagggatgctggctttcatattttgattactagctatcagctattagtcactgatgaaaagtattttcgccgggtgaagtggcaatatatggtgctagatgaggcccaagcaatcaagagttcctccagtataagatggaaaacccttcttagttttaactgtcggaaccgattgcttctgactggtactccaattcagaacaacatggcagagttatgggccctgctgcatttcatcatgccaatgttgtttgacaaccatgatcaatttaatgaatggttctcaaaaggaattgagaatcatgctgaacacggaggcactttaaatgagcaccagcttaacagactgcatgcgatcttgaaaccgttcatgcttcgacgggtaaaaaaggatgtggtttctgagctaactacaaagacggaagttacagtacactgcaagctcagttctcgacaacaagctttttatcaggctattaagaacaaaatttctctggctgagttgtttgatagcaaccgcggacaatttactgataagaaagtattgaatttaatgaatattgtcattcaactaaggaaggtttgcaaccatccagagttgttcgaaaggaatgaagggagctcgtatctctactttggagtgacttccaattctcttttgccccatccctttggtgagctagaggatgtacattattctggtggtcaaaatccgataatatacaagatacctaagctactacaccaagaggtgctccaaaattctgaaacattttgttcttctgtcgggcgtggcatctcaagagaatcttttctgaagcattttaatatatattcacctgagtatattcttaagtcaatattcccatctgatagtggggtagatcaagtggttagtggaagtggagcatttggcttttcacgcttgatggatctatcaccatcagaagttggatatctggctctgtgttctgttgcagaaaggctattattttctatactgaggtgggagcggcaatttttggatgaattagttaactctcttatggagtccaaggatggtgatcttagtgacaataacatcgagagagttaaaaccaaagctgtcacaagaatgttgctgatgccatcaaaagttgaaacgaattttcagaaaaggagactaagcacagggcctacccgtccttcatttgaagcgctagtgatctctcatcaggataggtttctttcaagtatcaaactcctgcattctgcatatacttatatcccaaaagccagagctccacctgtaagcattcattgctcggacagaaattcggcatacagagttacagaagaattacatcaaccatggcttaagagactattaatcggttttgcacgaacgtcagaagctaatggacccaggaagcctaacagctttccacatcctttaatccaagaaattgattcagaacttccagttgtgcagcctgcgcttcaactgacacacagaatatttggttcttgccctccaatgcaaagttttgacccagcaaagttgctcacggactctgggaagctgcagacacttgatatattattgaagcggcttcgagctggaaatcacagggtgctcctgtttgcacaaatgacaaagatgctgaacattctcgaggattatatgaactatagaaagtacaagtacctcaggcttgatggatcctccaccatcatggatcgccgagatatggttagggattttcagcataggagcgatatttttgtattcttgctgagcaccagagctggaggacttggtatcaacttgacggctgcagacactgtcattttctatgaaagtgattggaatcccaccttggatttacaagctatggacagggctcatcgtcttggacagacaaaagatggagacggtggaagagaaaattttgcacagggcaagtcagaaaaatacagttcaacagcttgttatgactggagggcatgttcagggtgatgattttcttggagctgcggatgtggtatctctgctaatggatgatgcggaggcagcacaactggagcagaaattcagagaactaccattacaggacaggcagaagaaaaagacgaaacgtatcagaatagatgctgaaggagatgcaactttggaagagttagaagatgttgaccgacaggataacggacaggaacctttggaagaaccggaaaagccaaaatccagtaataaaaagaggagagctgcttcaaatccgaaagctagagctcctcagaaagcaaaggaagaagcaaatggtgaagatactcctcagaggacaaaaagggtaaagagacaaacaaagagcataaacgaaagtcttgaacctgtattctctgcctctgtaacagaatcaaataaaggattcgatccaagtagctccgctaactaa

SEQ ID NO: 4 - DNA, 1377 bp, Arabidopsis thaliana
  CDS (1)...(1377): Rvbl (Atg22330)
atggagaaagtaaagattgaagaaattcagtccaccgctaagaaacaacggattgctactcacacccatatcaaaggccttggcctcgagccaactggtatccctataaaattggcagctggatttgttggtcaacttgaggctagagaggcagctggtcttgtagttgacatgattaagcagaagaaaatggcgggcaaggctcttttgcttgctggacctcctggaactgggaaaacagctttggctcttggaatctctcaagagctgggaagcaaggttccattctgtccaatggttggatctgaggtttactcatcagaggttaagaaaacagaggttctcatggagaattttagacgtgccattggtctacgtatcaaggaaaccaaagaagtctatgaaggggaggtcaccgagctgtcaccagaagaaactgaaagcctcactggaggttatggtaaaagcatcagccatgttgtaattacactcaagacagtcaaaggaaccaaacatctgaaattggatcccactatctatgatgccttgattaaggaaaaggtagctgtaggagatgtaatctatatcgaagcaaacagtggagctgtcaaacgggtaggtagaagtgatgcttttgccactgaatttgatctggaagcagaagaatatgttccacttcccaaaggagaggtccacaaaaagaaagagatagtgcaggatgtcacactccaagatctggatgcagcaaatgctcgacctcaaggtggccaggatatactttctttgatgggccaaatgatgaaaccgcggaagactgagatcactgataagcttcggcaagaaattaacaaggttgtgaaccgatatatagatgaaggtgtggcagagcttgttccaggagttctatttattgatgaggttcatatgcttgatatggagtgcttctcatacttgaaccgtgctcttgagagctcattatctccgatagtgatatttgcaacaaatagaggtgtttgcaacgtaagagggactgatatgcccagcccccatggagtccctattgatctattagatcgattggttatcatccggactcaaatctatgatccctctgaaatgatccagattatagccattcgtgcgcaagttgaagaattaaccgtggatgaagaatgcttggttctacttggggagattgggcaaagaacttcactaaggcacgctgtgcagcttctgtctcctgccagcattgtagcgaaaatgaatggccgtgacaatatttgcaaggctgatatagaggaagtaacatcactctacttggatgctaaatcttcagcaaagcttttgcatgagcaacaagaaaaatacatctcatga

SEQ ID NO: 5 - protein, 458 aa, Arabidopsis thaliana
MEKVKIEEIQSTAKKQRIATHTHIKGLGLEPTGIPIKLAAGFVGQLEAREAAGLVVDMIKQKKMAGKALLLAGPPGTGKTALALGISQELGSKVPFCPMVGSEVYSSEVKKTEVLMENFRRAIGLRIKETKEVYEGEVTELSPEETESLTGGYGKSISHVVITLKTVKGTKHLKLDPTIYDALIKEKVAVGDVIYIEANSGAVKRVGRSDAFATEFDLEAEEYVPLPKGEVHKKKEIVQDVTLQDLDAANARPQGGQDILSLMGQMMKPRKTEITDKLRQEINKVVNRYIDEGVAELVPGVLFIDEVHMLDMECFSYLNRALESSLSPIVIFATNRGVCNVRGTDMPSPHGVPIDLLDRLVIIRTQIYDPSEMIQIIAIRAQVEELTVDEECLVLLGEIGQRTSLRHAVQLLSPASIVAKMNGRDNICKADIEEVTSLYLDAKSSAKLLHEQQEKYIS

SEQ ID NO: 6 - DNA, 1584 bp, Arabidopsis thaliana
  CDS (1)...(1410): Rvb21 (At5g67630)
atggcggaactaaagctatcagagagtcgggacttaaccagagtcgagcgaatcggcgcacactcacacatcagaggactaggtctcgactctgccctcgagccgcgagctgtttccgaaggtatggtcggtcaagtgaaggcgcgtaaagccgccggtgtaatccttcagatgattagagaagggaaaatcgcgggtcgggctattctaatagcgggtcaacccggaacgggtaagacagcgattgcaatgggtatggcgaaatctcttggcttggaaactccttttgcgatgattgcaggaagtgaaattttctcattagagatgtcaaagacagaagctttgactcagtcttttcgtaaagcgattggtgttaggatcaaagaagagacagaggttattgaaggagaagttgttgaggttcagattgataggcctgcttcttctggtgttgcttccaagtcagggaagatgactatgaaaacgactgatatggaaactgtgtatgatatgggagctaagatgattgaggctttgaacaaggagaaagtgcagagtggtgatgttattgccattgataaagctactgggaagattactaagcttggaagatcgttttcgaggtctcgtgattatgatgctatgggtgcgcagaccaagtttgtgcagtgccctgaaggtgagttgcagaagaggaaagaggttgtacattgtgtcactcttcacgagattgatgttatcaacagcaggacacaagggtttcttgcccttttcactggcgatactggagaaatccgatcagaagtccgggaacaaattgatacaaaagtagctgaatggagagaagaaggaaaagcagagatagttcccggagttctcttcattgatgaagtccacatgctcgacatcgaatgcttctcattccttaaccgagctctagaaaacgaaatgtcaccaatccttgtggtggcaacaaaccgaggagtgacgacaatccgtggcacaaaccagaaatcaccacacgggatcccgattgatctccttgaccgtcttctcatcatcactacccaaccttacacagacgatgacataaggaagatattagaaatccgttgccaagaggaagacgttgagatgaacgaagaggccaaacagcttttgacattgatcggacgtgatacatctctaaggtatgcgattcatcttataaccgcagctgcattgtcatgccagaaacggaaagggaaagtcgtggaggttgaggatattcagagagtttacagactgttcttggatgtgaggagatcgatgcagtatcttgttgagtatcagagtcagtatatgttcagtgaaccaatcaaaaacgatgaagctgctgcagaagacgaacaagatgctatgcagatctgaggatccacctctgtttgccttatttatcatgtttcgtggtgatatgtatgattaggatgttgaactcggatttatgtttttttttttttaagttgtgacgagattcggttctagaaaatgatttaaccaagttcaatacagatcggtttggtacaaaacaaaaaaaaaaaaaaa

SEQ ID NO: 7 - protein, 469 aa, Arabidopsis thaliana
MAELKLSESRDLTRVERIGAHSHIRGLGLDSALEPRAVSEGMVGQVKARKAAGVILQMIREGKIAGRAILIAGQPGTGKTAIAMGMAKSLGLETPFAMIAGSEIFSLEMSKTEALTQSFRKAIGVRIKEETEVIEGEVVEVQIDRPASSGVASKSGKMTMKTTDMETVYDMGAKMIEALNKEKVQSGDVIAIDKATGKITKLGRSFSRSRDYDAMGAQTKFVQCPEGELQKRKEVVHCVTLHEIDVINSRTQGFLALFTGDTGEIRSEVREQIDTKVAEWREEGKAEIVPGVLFIDEVHMLDIECFSFLNRALENEMSPILVVATNRGVTTIRGTNQKSPHGIPIDLLDRLLIITTQPYTDDDIRKILEIRCQEEDVEMNEEAKQLLTLIGRDTSLRYAIHLITAAALSCQKRKGKVVEVEDIQRVYRLFLDVRRSMQYLVEYQSQYMFSEPIKNDEAAAEDEQDAMQI

SEQ ID NO: 8 - DNA, 1422 bp, Arabidopsis thaliana
  CDS (64)...(1389): Rvb22 (At3g49830)
atggcagaactaaggttatcagaaactcgagacttaactaggatcgaaagaatcggagcacactcacacatacgaggtttaggtctcgactcagtactcgagccacgagccgtatccgaaggaatggttggtcaaatcaaagcacgtaaagccgccggagtaaccctcgagttgatcagagacggcaaaatctcgggtcgggctatacttatagcgggtcaacccggaacgggtaaaatcgcaatagcaatgggtatagcaaaatcacttggacaagaaacaccattcactatgattgcaggaagtgagatcttttctttagagatgtcaaagactgaagctttaactcaagcttttcgtaaagctattggtgttaggatcaaagaagagactgacgtgatagaaggagaagttgtgacgatttcgattgatagacctgcttcttctggtggttctgtgaagaagactgggaagataacaatgaagacgactgatatggaatctaattttgatttgggatggaaattgattgagccattggataaggagaaagtacagagtggtgatgttattgttttggataggttttgtgggaagattactaagcttggaagatcttttacgaggtctagagattttgatgttatgggttcaaagactaagtttgtgcagtgccctgaaggtgagcttgagaagaggaaggaggttttgcattctgtcacacttcatgagattgatgttattaatagcaggactcaagggtatctagccctcttcacaggtgatacaggcgagattcgttcagaaacccgagagcaaagcgatactaaagtggcagagtggagagaagaagggaaagctgaaatagtacctggtgttctcttcattgatgaagtccatatgcttgatatcgaatgcttctctttcctgaatagagctctcgaaaacgatatgtcaccaatcctggtcgtggctacaaacagaggaatgacaacaatccgaggaacaaaccagatatcagcacatgggatcccaatcgattttcttgaccgtcttcttattatcacaacacagccttacacacaagacgagatcagaaatattttagagatccgttgccaagaagaggatgtggagatgaacgaggaagcgaaacagcttctgactttgatcggatgtaatacctcgcttaggtacgcgattcatctaatcaatgcagctgccctagcttgcctgaaacgtaaagggaaagtcgtagagattcaggacattgagagagtttatagattgtttttagacaccaagagatcgatgcagtacttggttgagcatgagagcgagtacttgtttagcgtgcctataaaaaacacacaggaggctactgcaggagaagaaacagaacacgaggccatggaagtttga

SEQ ID NO: 9 - protein, 442 aa, Arabidopsis thaliana
SHIRGLGLDSVLEPRAVSEGMVGQIKARKAAGVTLELIRDGKISGRAILIAGQPGTGKIAIAMGIAKSLGQETPFTMIAGSEIFSLEMSKTEALTQAFRKAIGVRIKEETDVIEGEVVTISIDRPASSGGSVKKTGKITMKTTDMESNFDLGWKLIEPLDKEKVQSGDVIVLDRFCGKITKLGRSFTRSRDFDVMGSKTKFVQCPEGELEKRKEVLHSVTLHEIDVINSRTQGYLALFTGDTGEIRSETREQSDTKVAEWREEGKAEIVPGVLFIDEVHMLDIECFSFLNRALENDMSPILVVATNRGMTTIRGTNQISAHGIPIDFLDRLLIITTQPYTQDEIRNILEIRCQEEDVEMNEEAKQLLTLIGCNTSLRYAIHLINAAALACLKRKGKVVEIQDIERVYRLFLDTKRSMQYLVEHESEYLFSVPIKNTQEATAG

SEQ ID NO: 10 - DNA, 1591 bp, Arabidopsis thaliana
  gene (1)...(1591): At3g57290
ccacgcgtccgtaagaagattttgccagtgcgggaagcggcggagattgagagattagcgacgatggaggaaagcaaacagaactatgacctgacgccactaatagcgcctaacctggacagacacttggtgtttcctatattcgagttccttcaagagcgtcagctttaccctgatgagcagatcctgaagtctaaaatccagcttttgaaccagacgaacatggttgattacgccatggatattcacaagagtctctaccacactgaagacgctcctcaagaaatggtggagagaagaacagaggttgtcgctaggctcaaatctttggaggaggctgctgcaccactcgtgtcttttcttttgaaccctaacgctgtgcaggagctaagagctgacaagcagtacaatctccaaatgctcaaggaacgctaccagattggtccagaccagattgaggctttgtaccagtacgccaagtttcagtttgaatgtggcaactattctggtgctgctgattatctttaccagtacaggaccctgtgctctaaccttgagaggagtttgagtgccttgtggggaaagctcgcatctgaaatattgatgcaaaactgggatattgctcttgaagagcttaaccgtctcaaagagattattgactcaaagagtttttcatcgccgttaaaccaggtgcagaacaggatttggttgatgcattggggtctgtatatcttttttaaccatgataatggaaggacacagatcattgatctttttaaccaagacaagtatctgaatgccatccaaactagtgctccacacttgctgcgctacttggcaactgctttcattgtcaacaaaaggagaagaccacaattgaaagaattcattaaggtcattcagcaagagcactactcctacaaagatccaattatcgagttcctggcatgtgtgtttgtcaattatgactttgatggggctcaaaagaagatgaaagagtgtgaagaggtcattgtgaatgatccattccttggcaagcgagttgaggatggaaacttttcaactgtaccactgagagatgaatttcttgaaaatgcccgcctattcgtctttgaaacctattgcaaaattcatcaaaggattgacatgggggtacttgctgaaaaattgaatctgaactatgaggaggccgagagatggattgtgaacctaatccgcacctcaaagcttgatgccaagattgattctgagtcaggaactgtaatcatggagcctactcagcccaacgtgcatgagcagttgataaaccacaccaaaggcttatcaggacgaacatacaagttagtgaatcagctcttggaacacacacaggcgcaagcaactcgctagtcaaaattttgctgtggaagcctttccttgataaaactcaccttcggttgactggaattattttctttttcttgctctgagttcaccttttactttgaaaaagattattatggagttgttctatttgaaatgttggatccacagattggaacattttccaaccaaatcagcattttgtaaaaaaaaaaaaaaaaaaaaaaaa

SEQ ID NO: 11 - DNA, 1773 bp, Arabidopsis thaliana
  CDS (1)...(1773): AtArp5.1 (At3g12380)
atggcagaactgctatttgagacttatggagtgcctgcagttgcatttggagtcgatgctgctttcagctacaaatacaatcaactacatggaatttgtaaaaaagatggaattgttctctgtcctggattcacgacaacacactccattccgtttgtcgacggagaacctatatataaaggatccagccgaactaacattggtggatatcatgtcactgattatttaaagcagcttctgtcacttaagtacccttttcattcgtctaggtttacatgggagaaggccgaagatttgaaattggaacactgttatatcgcacctgactatgcttcggaaattcggttattccaggaaggaagaaaagaagctgaagagaaaacaagttattggcagcttccatggatacctcctcccaccgaagttcctccatcagaagaagagattgcaaggaaggcagctataagagaaaaacaaggtcaaaggctgcgagaaatggctgaagcaaagagagtgtccaagattaatgacatggagaatcaactgattagcttgcgtttccttttgaagcaagttgaccaggttgaagaggatgatattccaacctttttgtcagacaccggttacgcgtccaggcaagagctagagtctactattacgaaagtgacacagtcgcttagaaaagcaaggggtgagccgaagaatgaaccagctgagtatgaagaaaaccctgattctcttaataatgaaaagtatccacttatgaatgtccccgatgatattcttactcctgagcagcttaaggacaagaagaggcaaatgtttcttaaaacaacagcagagggccggctacgagctagacaaaagcgtaatgaggaggaactcgaaaaagagaaaagaaatcaattagaggaggaaagacgtcgtgagaacccagagtcttacttagaggagttgcaagctcagtacaaggaagtgttggagagagttgagcagaagaagcgtctgaaaacaaacgggtccagtaacgggaataacaagtctggaggtattgggcgaggcgagcgactcagtgctgcacagagggagagaatgcgtctgctgacgacagcagcctttgatagagggaaaggcgaggatacgtttggttctagagatgaagattggcagctctacaaacttatgagcaaggataatgacgatgatgacgaacaacctgattcagacgaggcagagttggctcgtttatcatctagacttcaggaaattgatccaacatttgtgcagaaagtagaaggagaattgagtcagacatcaggggaggtgccacgcgtacgcccattaacagaggaagactacaagatagtgattggtatagaaagattccgttgcccagagatcctgttccatccaaaccttattggtattgaccaagtaggcttagacgagatggctggcacatcaatcagaaggctaccgcacgacgagaaagagttagaggagaggctaacgagctcgatactaatgacgggcgggtgtagccttcttccagggatgaacgagcggttggaatgtgggattaggatgataagaccttgcggatcacccattaacgtggttagagctatggatccagtgctggatgcatggcgaggagcatctgcatttgctgctaatttgaacttcttggggaatgcctttactaagatggattacgacgagaaaggtgaagattggcttagaaattatcaaattcgatacaactatttgtga

SEQ ID NO: 12 - protein, 590 aa, Arabidopsis thaliana
MAELLFETYGVPAVAFGVDAAFSYKYNQLHGICKKDGIVLCPGFTTTHSIPFVDGEPIYKGSSRTNIGGYHVTDYLKQLLSLKYPFHSSRFTWEKAEDLKLEHCYIAPDYASEIRLFQEGRKEAEEKTSYWQLPWIPPPTEVPPSEEEIARKAAIREKQGQRLREMAEAKRVSKINDMENQLISLRFLLKQVDQVEEDDIPTFLSDTGYASRQELESTITKVTQSLRKARGEPKNEPAEYEENPDSLNNEKYPLMNVPDDILTPEQLKDKKRQMFLKTTAEGRLRARQKRNEEELEKEKRNQLEEERRRENPESYLEELQAQYKEVLERVEQKKRLKTNGSSNGNNKSGGIGRGERLSAAQRERMRLLTTAAFDRGKGEDTFGSRDEDWQLYKLMSKDNDDDDEQPDSDEAELARLSSRLQEIDPTFVQKVEGELSQTSGEVPRVRPLTEEDYKIVIGIERFRCPEILFHPNLIGIDQVGLDEMAGTSIRRLPHDEKELEERLTSSILMTGGCSLLPGMNERLECGIRMIRPCGSPINVVRAMDPVLDAWRGASAFAANLNFLGNAFTKMDYDEKGEDWLRNYQIRYNYL

SEQ ID NO: 13 - DNA, 1416 bp, Arabidopsis thaliana
  CDS (1)...(1416): AtArp5.2 (At5g56180)
atgatcctgaagaaagtatggggatcggtgtggaaccgatcgaatagtggcaaggatttggtgaatcatcagagagcgatcgatgttcctcctctgttattgtcttcttcatcgtctcttggtgcgtttgatcagctaccgatggatattctagtccagatactgatgatgatggagccaaaagatgctgtgaaattgggcttaacgtgcaaagcctggaaatgcgtagctagtggtaatcgtctctggatattttatctccagtgttctcaagagccatgggactccattttcttcgctgaaactagtttgcgttctggttatcctctccgaatgatttctagtcaatcaggagagttgtcgtttatgcacatttatagtcagagggcacaagttcctggttctatcattattgatggtggttctggatattgtaagtttggttggagcaagtatgcgtctccttctggacgttctgctacttttttggaatttggtaacattgagtcaccgatttatgctagacttcaacagttctttgcaaccattttcaccaggatgcaggtaaagccctctatgcagccaatagtggtatcactacctctctgccattttgatgatactgaatcagccaaggcatcaaggcggcaacttaagactgctattttcaatgtcttgtttgacatgaacgtccctgcagtgtgtgcagttaatcaggctgtgttagctctatatgcagcacggcggacatctggaattgttgtcaacattggtttccaagtcatcaccattcttccgattttacatggtaaggtgatgcgccaggtaggtgtagaagtcattggttttggagcattgaaactcacgggcttccttaaggagaagatgcaagaaaacaacatttcctttcaatcactctacactgttcgtactcttaaagagaaactgtgttatgtggctcttgattataaagctgaactttcaaaagacacacaagcttcagtggaagtttcaggtgaaggatggtttactttatcaaaagagcgtttccaaacaggggagatattattccaaccacgtctcgctggaatgcgtgcaatgagtctgcaccaggccgtctcgctctgtatggaccactgtgatgcagcaggacttacaggtgacgatagttggttcaagactgtagtactaactgggggaagcgcgtgtttgcctggactctcagagaggctagagagagaactgcaagatcaccttccttcatctataagtaacggaatcagagtaatacctcctccttacggcgtggacacatcatggcatggggcaaagcttattagtaatttgagcatctttcctggtccttggtgtatcacaaggaagcagttccgtcgcaagtcaagactcatgtggtga

SEQ ID NO: 14 - protein, 471 aa, Arabidopsis thaliana
MILKKVWGSVWNRSNSGKDLVNHQRAIDVPPLLLSSSSSLGAFDQLPMDILVQILMMMEPKDAVKLGLTCKAWKCVASGNRLWIFYLQCSQEPWDSIFFAETSLRSGYPLRMISSQSGELSFMHIYSQRAQVPGSIIIDGGSGYCKFGWSKYASPSGRSATFLEFGNIESPIYARLQQFFATIFTRMQVKPSMQPIVVSLPLCHFDDTESAKASRRQLKTAIFNVLFDMNVPAVCAVNQAVLALYAARRTSGIVVNIGFQVITILPILHGKVMRQVGVEVIGFGALKLTGFLKEKMQENNISFQSLYTVRTLKEKLCYVALDYKAELSKDTQASVEVSGEGWFTLSKERFQTGEILFQPRLAGMRAMSLHQAVSLCMDHCDAAGLTGDDSWFKTVVLTGGSACLPGLSERLERELQDHLPSSISNGIRVIPPPYGVDTSWHGAKLISNLSIFPGPWCITRKQFRRKSRLMW

SEQ ID NO: 15 - DNA, 1092 bp, Arabidopsis thaliana
  CDS (1)...(1092): AtArp5.3 (At3g60830)
atggaagcactagttgttgatgctggctctaagttcctgaaagcaggagcagcaattcctgaccagtctcctgcaatgataattccctctcaaatgaaacgaatggttgatgatgggtcttcttcagctgataaccccaccactgtctttgaggatgtcactcttgatcctattgaaaggggtttgattagagattgggatgctatggaagatctgttgcgttatgttgtctacactgggcttggatgggaagagggaaacgaaggcaatatactttttacagatccactttgtactcctaaggctattagggagcaattggtgcagttgatgtttgaaacattcaatgtctctggattttatgcatctgagcaagcagtgttgtccctttatgctgttggacgcatctccggttgcactgttgatattggtcatgggaagatagatattgccccagttcttgaaggtgcagtacaacacattgcctcgaaacggtttgagctaggtggaaccgagctaactaaattatttgcccaagagcttggaaaaaccaacccgtcgatgaatctcagcatgtctgatgttgaaaaactcaaggagcagtatgcaaactgtgccgaggacgaaattgcttacaaaaaaacccaaaactgtgaaatcgagcagcatactcttcctgatggacaggtgataagcatcgggcgagagagatactcggttggagaagctctgtttcagccatcaatactgggactggaggagcatggaatcgttgagcagcttgtccggattatctccacagtgtcatctgagaaccataggcagctcttggagaacactgtactttgtggtggtacaacctccatgacaggattcgaaagtagattccagaaagaagcaaacttgtgctcatctgccattaggccaacactggtgaaaccgccagaatatatgccggagaatttggggatgtattcggcttgggttggaggagccatactagctaaagtggtgtttccgcagaatcagcacgttactaaagcagattatgacgagactggaccatcagtggttcacaggaaatgtttctga

SEQ ID NO: 16 - protein, 363 aa, Arabidopsis thaliana
MEALVVDAGSKFLKAGAAIPDQSPAMIIPSQMKRMVDDGSSSADNPTTVFEDVTLDPIERGLIRDWDAMEDLLRYVVYTGLGWEEGNEGNILFTDPLCTPKAIREQLVQLMFETFNVSGFYASEQAVLSLYAVGRISGCTVDIGHGKIDIAPVLEGAVQHIASKRFELGGTELTKLFAQELGKTNPSMNLSMSDVEKLKEQYANCAEDEIAYKKTQNCEIEQHTLPDGQVISIGRERYSVGEALFQPSILGLEEHGIVEQLVRIISTVSSENHRQLLENTVLCGGTTSMTGFESRFQKEANLCSSAIRPTLVKPPEYMPENLGMYSAWVGGAILAKVVFPQNQHVTKADYDETGPSVVHRKCF

SEQ ID NO: 17 - DNA, 1791 bp, Arabidopsis thaliana
  CDS (1)...(1791): AtArp8 (At5g43500)
atggcaaaaccaaaatccaattctcatctctcgtggcaggattacttgaaaactgttgcccccactcagattttgtctgaacgaggagctaatctcgttgtcatcaacttagggtctgctaatgtaagagtaggattggctatggatgagaaacctttcaatgttcctaattgtattgcaagatacattacacaatctgggaaaccaactgttgttgatcagatgcttaacactgaggtcactactaatcaacatgttgaccgtgagagagcttataattctgcggcatcgctgttgaagatactgttcctggatgaaagttcttcttctggctctgcatctcgcaagatggggaggatcgacggatacaatcaagcgagtacgataaagaaagattcagtctttacttggactgatgtgtatgaggatgagaaaatttctcttgcctcaccagcagaaacttctcctgataaaggtgatgctagtgcatctgaagctgttcctgatgttactgactctaaagatactagtgagagtaaacgcaagtataggaaaatgatttttggtgaagaagctttgaaaatatcgccaaaagagccatattgtttataccatcctattcggagaggtcacttcaatgtttcgccacattattcagcgcaacgggtttgtgaggacttaactgctatcttggactggattttattagagaaacttcatataactcacaaggagagattctcttttcatgctgttattgttgtcccagaaacatttgacacccgcgaaataaaggaaatgctaactattgtgttgggagagctatactttaactcagcagttgtccaccaagaaggtctatcggccgtttttgggaatggtttgacaacagcttgtattgtgaatataggagcccagacaagtacagtagtttgtgtcgaggatggggtctcattgccaaatactgaaaagattttaccttttggaggagaggatatatgtagatgccttctatggattcagaggcattaccaaaagtggccacaaatcaacacagatgttttggcaaagccaatcgatatgctgatgcttaatcaacttaaggagtcattttgtgaaattagagcaggagaacttgaaactgttgcaacggttcattcttatgaggaaggcatgccagctgtgcctcacaagacaaatctcacctcccttaacgttccaccaatgggtctgttttatcctaaccttttggtccctgaaatatttccccagccaccacgtcaatggtttcaagactacgagaatatgttggaagacacttggaacatggactttggaggtggtggtaacatgggattaccaatgtgggatagttttgcattttcgccttcaaaaccaaagaaagaagagaagattggtctcgctgaagccattacaagcagcattctctctgctggacgcatagaccttagacggaagcttttctccagcattcaattgattggtggtgctggtttgacgaaaggtcttgtagcggcagtggaagaaagagttcttcacgcgatacctccaactgaagccattgatacagtgcaggttctgccatcaagaacggagccacaattcgtaacttggaaaggaggagcgatattgggaattctggactttgggagggaggcttggattgagagacatcaatggatggtaaatggggttaataaaggtggtctaaagaagtacaaagactcttatcaccttcaaggtcaagcaatgtacttcatcaacccctag

SEQ ID NO: 18 - protein, 596 aa, Arabidopsis thaliana
MAKPKSNSHLSWQDYLKTVAPTQILSERGANLVVINLGSANVRVGLAMDEKPFNVPNCIARYITQSGKPTVVDQMLNTEVTTNQHVDRERAYNSAASLLKILFLDESSSSGSASRKMGRIDGYNQASTIKKDSVFTWTDVYEDEKISLASPAETSPDKGDASASEAVPDVTDSKDTSESKRKYRKMIFGEEALKISPKEPYCLYHPIRRGHFNVSPHYSAQRVCEDLTAILDWILLEKLHITHKERFSFHAVIVVPETFDTREIKEMLTIVLGELYFNSAVVHQEGLSAVFGNGLTTACIVNIGAQTSTVVCVEDGVSLPNTEKILPFGGEDICRCLLWIQRHYQKWPQINTDVLAKPIDMLMLNQLKESFCEIRAGELETVATVHSYEEGMPAVPHKTNLTSLNVPPMGLFYPNLLVPEIFPQPPRQWFQDYENMLEDTWNMDFGGGGNMGLPMWDSFAFSPSKPKKEEKIGLAEAITSSILSAGRIDLRRKLFSSIQLIGGAGLTKGLVAAVEERVLHAIPPTEAIDTVQVLPSRTEPQFVTWKGGAILGILDFGREAWIERHQWMVNGVNKGGLKKYKDSYHLQGQAMYFINP

SEQ ID NO: 19 - DNA, 29 bp, Arabidopsis sp.
  protein_bind (1)...(29): sm5UT primer
ctagaagcttttaaggattaagactctcc

SEQ ID NO: 20 - DNA, 22 bp, Arabidopsis sp.
  primer_bind (1)...(22): 3′ primer
ctcgtatgtatcccccttctcc

SEQ ID NO: 21 - DNA, 21 bp, Arabidopsis thaliana
  primer_bind (1)...(21): 5′ At3g57300 primer for semi-quantitative RT-PCR
tgatggatctatcaccatcag

SEQ ID NO: 22 - DNA, 21 bp, Arabidopsis thaliana
  primer_bind (1)...(21): 3′At3g57300 primer for semi-quantitative RT-PCR
ggtgggattccaatcactttc

SEQ ID NO: 23 - DNA, 21 bp, Arabidopsis thaliana
  primer_bind (1)...(21): Atino80 primer for semi-quantitative RT-PCR
tgatggatctatcaccatcag

SEQ ID NO: 24 - DNA, 21 bp, Arabidopsis thaliana
  primer_bind (1)...(21): Atino80 primer for semi-quantitative RT-PCR
ggtgggattccaatcactttc

SEQ ID NO: 25 - DNA, 19 bp, Arabidopsis thaliana
  primer_bind (1)...(19): AtRvb1 primer for semi-quantitative RT-PCR
tttgatgggccaaatgatg

SEQ ID NO: 26 - DNA, 36 bp, Arabidopsis thaliana
  primer_bind (1)...(36): AtRvb1 primer for semi-quantitative RT-PCR
cttccaacctaggtgagatgtttcaacaaaatgtgc

SEQ ID NO: 27 - DNA, 19 bp, Arabidopsis thaliana
  primer_bind (1)...(19): AtF1vb21 primer for semi-quantitative RT-PCR
tcaacagcaggacacaagg

SEQ ID NO: 28 - DNA, 37 bp, Arabidopsis thaliana
  primer_bind (1)...(37): AtF1vb21 primer for semi-quantitative RT-PCR
cccaatgcctaggaaatccgagttcaacatcctaatc

SEQ ID NO: 29 - DNA, 23 bp, Arabidopsis thaliana
  primer_bind (1)...(23): AtRvb22 primer for semi-quantitative RT-PCR
acaaaccagatatcagcacatgg

SEQ ID NO: 30 - DNA, 23 bp, Arabidopsis thaliana
  primer_bind (1)...(23): AtRvb22 primer for semi-quantitative RT-PCR
aacaagtactcgctctcatgctc

(19) United States
(12) Patent Application Publication
(10) Pub. No.: US 2006/0121561 A1
(43) Pub. Date: Jun. 8, 2006

(54) Gene for increased somatic recombination

(76) Inventors: Olivier Fritsch, Colmar (FR); Barbara Hohn, Arlesheim (CH); Jan Martin Lucht, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/519,069

(22) PCT Filed: Jun. 26, 2003
(86) PCT No.: PCT/EP03/06757

§ 371 Date: Dec. 22, 2004

(30) Foreign Application Priority Data
Jun. 27, 2002 (GB) 0214896.3

(51) Int. Cl. C12P 21/06; C07H 21/04; C12N 9/22; C12N 15/87

(52) U.S. Cl. 435/69.1; 536/23.2; 435/199; 435/455; 435/325

(57) ABSTRACT

The present invention relates to nucleic acids encoding polypeptides involved in homologous recombination, as well as vectors and host cells comprising the nucleic acids and polypeptides encoded by the nucleic acids. Also provided are methods for inducing somatic and/or meiotic homologous recombination in a cell, comprising modulating the expression or properties of one or more gene products selected from the group consisting of AtIno80, At3g57300, Rvb1 (At5g22330), Rvb21 (At5g67630), Rvb22 (At3g49830), At3g57290, AtArp5.1 (At3g12380), AtArp5.2 (At5g56180), AtArp5.3 (At3g60830) and AtArp8 (At5g43500), their homologues, fragments or derivatives. In particular, the methods can be used to increase gene targeting.

### TECHNICAL FIELD

The present invention relates to DNA that encodes proteins that control somatic recombination, in particular in plants.

### BACKGROUND

Cells of all organisms have evolved a series of DNA repair pathways that counteract the deleterious effects of DNA damage and are triggered by intricate signal cascades. Homologous recombination in plants stabilizes the genome by repairing damaged chromosomes simultaneously generating genetic variability through the creation of new genes and new genetic linkages. Repair of DNA damage by recombination is particularly significant for cells under exogenous and endogenous genotoxic stress because of its potential to remove a wide range of DNA lesions. The current understanding of genetic and molecular components underlying meiotic and somatic recombination and DNA repair in plants is limited. To be able to modify or improve DNA repair using gene technology it is necessary to identify key proteins involved in said pathways or cascades.

The precise manipulation of the genome of higher plants is still a major challenge for plant genetic engineering. Some advances have been made recently for the creation of point mutations at predetermined positions by chimeric RNA/DNA oligonucleotides (Beetham et al. 1999, Hohn & Puchta 1999, Zhu et al. 1999, Kipp et al. 2000, Zhu et al. 2000). However, the targeted insertion of longer stretches of DNA sequence at any desired location (“knock-in”) or the replacement of predetermined plant genomic sequences by heterologous DNA (“knock-out) via homologous recombination is at present not possible as a routine technique (Mengiste & Paszkowski 1999, Puchta 2002).

Few reports have appeared in the literature that describe successful “gene targeting” in higher plants (Paszkowski et al. 1988, Lee et al. 1990, Offringa et al. 1990, Miao & Lam 1995, Kempin et al. 1997, Hanin et al. 2001), but the reported absolute numbers and relative frequencies of the desired events were very low. Indeed, the main problem for “gene targeting” experiments is the low frequency of the desired homologous recombination events—10⁻³ to 10⁻⁵ (Hohn & Puchta 1999, Mengiste & Paszkowski 1999)—relative to illegitimate recombination/integration events.

Various attempts of increasing the low relative frequency of targeted homologous recombination events, by improved selection schemes (“positive-negative selection”) or by providing extended regions of sequence homology, were not successful (Thykjaer et al. 1997, Gallego et al. 1999). One promising strategy to facilitate gene targeting in higher plants would be to shift the balance between illegitimate and homologous recombination events towards the latter, by facilitating homologous recombination events in plants by genetic manipulation (Gherbi et al. 2001).

One approach described in the literature is the expression in plants of heterologous proteins known to be involved in homologous recombination. Overproduction of the bacterial resolvase RuvC was shown to increase somatic inter-and intra-chromosomal recombination, as well as extrachromosomal recombination (Shalev et al. 1999), but no gene targeting studies were reported yet with this system. Expression of the bacterial RecA protein had similar effects (Reiss et al. 1996, Reiss et al. 1997), but subsequent experiments did not show an increase of gene targeting events (Reiss et al. 2000). So far, it is not clear whether heterologous proteins can successfully interact with the plant recombination machinery to affect the outcome of the recombination events required for gene targeting. In addition, these foreign proteins might have undesired side effects in plants.

An alternative approach is to rely on endogenous plant genes to influence the frequency of homologous recombination events. So far, indirect approaches have been reported to isolate plant genes involved in recombination. The cloning of plant orthologs to recombination and repair genes from other species was reported (Klimyuk & Jones 1997, Doutriaux et al. 1998, Hartung & Puchta 1999, Gallego et al. 2000, Lin et al. 2000), but so far the importance of these genes for recombination in plants has only been evaluated for the RAD50 homologue (Gherbi et al., 2001). Functional screens have been carried out to identify plant mutants hypersensitive to genotoxic treatments (Davies et al. 1994, Jenkins et al. 1995, Jiang et al. 1997, Masson et al. 1997, Albinsky et al. 1999, Mengiste et al. 1999). Since recombination is an important mechanism for DNA repair, some of these mutants might be affected in their recombination behavior. This was experimentally demonstrated for some X-ray hypersensitive Arabidopsis mutants that also showed reduced levels of somatic recombination (Masson & Paszkowski 1997), although the affected gene has not been isolated. Recently, a DNA damage hypersensitive Arabidopsis mutant was isolated from a T-DNA tagged population, the affected gene (MIM) was cloned and shown to encode an SMC (Structural Maintenance of Chromatin) protein. Since the mim mutant showed decreased frequencies of somatic recombination, MIM seems be involved in some aspect of somatic recombination (Mengiste et al. 1999). Also in tobacco a hyperrecombinogenic mutant was isolated (Gorbunova et al. 2000). However, the gene affected could not be isolated so far.

Previously, a genetic system was described to study somatic homologous recombination between repeated sequences in whole plants (Swoboda et al. 1994, Puchta et al. 1995a, Puchta et al. 1995b). Briefly, a transgene carrying two non-functional halves of the β-glucuronidase reporter gene sharing a stretch of sequence identity serves as a reporter construct. Homologous recombination between the repeated sequences results in the restoration of a functional reporter gene. Such events were detected by a sensitive histochemical assay, and confirmed by Southern blotting. This assay is destructive, since the staining procedure is lethal, so that direct isolation of mutants is difficult.

There is a need in the art to identify genes that increase somatic recombination and this invention meets that need.

### RELATED LITERATURE

1) Aguilera, A. & Klein, H. L. Genetic control of intrachromosomal recombination in Saccharomyces cerevisisae. I. Isolation and genetic characterization of hyper-recombination mutations. Genetics 119: 779-790, 1985.
2) Albinsky, D. et al. Plant responses to genotoxic stress are linked to an ABA/salinity signaling pathway. Plant J 17:73-82, 1999.
3) Aravind, L. et al. Conserved domains in DNA repair proteins and evolution of repair systems. Nucl Acids Res 27:1223-1242, 1999.
4) Beetham, P. R. et al. A tool for functional plant genomics: chimeric RNA/DNA oligonucleotides cause in vivo gene-specific mutations. Proc Natl Acad Sci USA 96: 8774-8778, 1999.
5) Cho, S.-G. et al. TIP49b, a regulator of activating transcription factor 2 response to stress and DNA damage. Mol Cell Biol 21:8398-8413, 2001.
6) Clough, S. J. & Bent, A. F. Floral dip: a simplified method for Agrobacterium-mediated transformation of Arabidopsis thaliana. Plant J 16:735-743, 1998.
7) Davies, C. et al. Isolation of Arabidopsis thaliana mutants hypersensitive to gamma radiation. Mol Gen Genet 243:660-665, 1994.
8) Davis, J. L. et al. A presumptive helicase (MOT1 gene product) affects gene expression and is required for viability in the yeast Saccharomyces cerevisiae. Mol Cell Biol 12:1879-1892, 1992.
9) Dilkes, B. P. & Feldmann, K. A. Cloning genes from T-DNA tagged mutants. Methods Mol Biol 82:339-351, 1998.
10) Doutriaux, M.-P. et al. Isolation and characterisation of the RAD51 and DMC1 homologs from Arabidopsis thaliana. Mol Gen Genet 257:283-291, 1998.
11) Ebbert, R. et al. The product of the SNF2/SWI2 paralogue INO80 of Saccharomyces cerevisiae required for efficient expression of various yeast structural genes is part of a high-molecular-weight protein complex. Mol Microbiol 32:741-751, 1999.
12) Emery, H. S. et al. Sequence of RAD54, a Saccharomyces cerevisiae gene involved in recombination and repair. Gene 104:103-106, 1991.
13) Essers, J. et al. Homologous and non-homologous recombination differentially affect DNA damage repair in mice. EMBO J. 19:1703-1710, 2000.
14) Fang, R. X. et al. Multiple cis regulatory elements for maximal expression of the cauliflower mosaic virus 35S promoter in transgenic plants. Plant Cell 1:141-150, 1989.
15) Fridborg, I. et al. The Arabidopsis dwarf mutant shi exhibits reduced gibberellin responses conferred by overexpression of a new putative zinc finger protein. Plant Cell 11:1019-1032, 1999.
16) Gallego, F. et al. AtRAD1, a plant homologue of human and yeast nucleotide excision repair endonucleases, is involved in dark repair of UV damages and recombination. Plant J 21:507-518, 2000.
17) Gallego, M. E. et al. Positive-negative selection and T-DNA stability in Arabidopsis transformation. Plant Mol Biol 39:83-93, 1999.
18) Galli, A. et al. Characterization of the hyperrecombination phenotype of the pol3-t mutation of Saccharomyces cerevisiae. Genetics 164: 65-79, 2003.
19) Gamborg, O. L. et al. Nutrient requirement of suspension cultures of soybean root cells. Exp Cell Res 50:151-158, 1968.
20) Gherbi, H. et al. Homologous recombination in planta is stimulated in the absence of Rad50. EMBO Rep 2:287-291, 2001.
21) Gorbalenya, A. E. & Koonin, E. V. Helicases: amino acid sequence comparisons and structure-function relationships. Curr Opin Struct Biol 3:419-429, 1993.
22) Gorbunova, V. et al. A new hyperrecombinogenic mutant of Nicotiana tabacum. Plant J 24:601-611, 2000.
23) Guerineau, F. et al. Sulfonamide resistance gene for plant transformation. Plant Mol Biol 15:127-136, 1990.
24) Hanin, M. et al. Gene targeting in Arabidopsis. Plant J 28:671-677, 2001.
25) Hartung, F. & Puchta, H. Isolation of the complete cDNA of the Mre11 homologue of Arabidopsis (Accession No. AJ243822) indicates conservation of DNA recombination mechanisms between plants and other eucaryotes. (PGR99-132). Plant Physiol 121: 312, 1999.
26) Hayashi, H. et al. Activation of a plant gene by T-DNA tagging: auxinindependent growth in vitro [retracted by Schell J. Science 284:1275, 1999]. Science 258:1350-1353, 1992.
27) Hohn, B. & Puchta, H. Gene therapy in plants. Proc Natl Acad Sci USA 96:8321-8323, 1999.
28) Ikura, T. et al. Involvement of the TIP60 histone acetylase complex in DNA repair and apoptosis. Cell 102:463-473, 2000.
29) Jenkins, M. E. et al. Radiation-sensitive mutants of Arabidopsis thaliana. Genetics 140: 725-732, 1995.
30) Jelesko J. G. et al. Rare germinal unequal crossing-over leading to recombinant gene formation and gene duplication in Arabidopsis thaliana. Proc Natl Acad Sci USA 96:10302-10307, 1999.
31) Jiang, C.-Z. et al. UV- and gamma-radiation sensitive mutants of Arabidopsis thaliana. Genetics 147:1401-1409, 1997.
32) Jonsson, Z. O. et al. Rvb1p and Rvb2p are essential components of a chromatin remodeling complex that regulates transcription of over 5% of yeast genes. J Biol Chem 276:16279-16288, 2001.
33) Kakimoto, T. CKI1, a histidine kinase homolog implicated in cytokinin signal transduction. Science 274:9825, 1996.
34) Kanemaki, M. et al. TIP49b, a new RuvB-like DNA helicase, is induced in a complex together with another RuvB-like DNA helicase, TIP49a. J Biol Chem 274:22437-22444, 1999.
35) Kardailsky, I. et al. Activation tagging of the floral inducer FT. Science 286:1962-1965, 1999.
36) Kempin, S. A. et al. Targeted disruption in Arabidopsis. Nature 389:802-803, 1997.
37) Kipp, P. B. et al. Gene targeting in plants via site-directed mutagenesis. Methods Mol Biol 133:213-221, 2000.
38) Klimyuk, V. I. & Jones, J. D. AtDMC1, the Arabidopsis homologue of the yeast DMC1 gene: characterization, transposon-induced allelic variation and meiosis-associated expression. Plant J 11:1-14, 1997.
39) Laurent, B. C. et al. Functional interdependence of the yeast SNF2, SNF5, and SNF6 proteins in transcriptional activation. Proc Natl Acad Sci USA 88:2687-2691, 1991.
40) Lee, K. Y. et al. Homologous recombination in plant cells after Agrobacterium-mediated transformation. Plant Cell 2:415-425, 1990.
41) Liu, Z. et al. Repair of UV damage in plants by nucleotide excision repair: Arabidopsis UVH1 DNA repair gene is a homolog of Saccharomyces cerevisiae Rad1. Plant J 21:519-528, 2000.
42) Masson, J. E. et al. Mutants of Arabidopsis thaliana hypersensitive to DNA-damaging treatments. Genetics 146:401-407, 1997.
43) Masson, J. E. & Paszkowski, J. Arabidopsis thaliana mutants altered in homologous recombination. Proc Natl Acad Sci USA 94:11731-11735, 1997.
44) Mathur, J. et al. Gene identification with sequenced T-DNA tags generated by transformation of Arabidopsis cell suspension. Plant J 13:707-716, 1998.
45) Mayerhofer, R. et al. T-DNA integration: a mode of illegitimate recombination in plants. EMBO J. 10:697-704, 1991.
46) Mengiste, T. & Paszkowski, J. Prospects for the precise engineering of plant genomes by homologous recombination. Biol Chem 380:749-758, 1999.
47) Mengiste, T. et al. An SMC-like protein is required for efficient homologous recombination in Arabidopsis. EMBO J. 18:4505-4512, 1999.
48) Miao, Z. H. & Lam, E. Targeted disruption of the TGA3 locus in Arabidopsis thaliana. Plant J 7:359-365, 1995.
49) Michelet, B. & Chua, N.-H. Improvement of Arabidopsis mutant screens based on luciferase imaging in planta. Plant Mol Biol Rep 14:320-329, 1996.
50) Millar, A. J. Circadian clock mutants in Arabidopsis identified by luciferase imaging. Science 267:1161-1163, 1995a.
51) Millar, A. J. et al. Firefly luciferase as a reporter of regulated gene expression in higher plants. Plant Mol Biol Rep 10:324-327, 1992.
52) Millar, A. J. et al. The regulation of circadian period by phototransduction pathways in Arabidopsis. Science 267:1163-1166, 1995b.
53) Muchardt, C. & Yaniv, M. ATP-dependent chromatin remodelling: SWI/SNF and Co. are on the job. J Mol Biol 293:187-98, 1999.
54) Muris, D. P. et al. Isolation of the Schizosaccharomyces pombe RAD54 homologue, rhp54+, a gene involved in the repair of radiation damage and replication fidelity. J Cell Sci 109:73-81, 1996.
55) Nacry, P. et al. Major chromosomal rearrangements induced by T-DNA transformation in Arabidopsis. Genetics 149:641-650, 1998.
56) Offringa, R. et al. Extrachromosomal homologous recombination and gene targeting in plant cells after Agrobacterium mediated transformation. EMBO J. 9:3077-3084, 1990.
57) Paszkowski, J. et al. Gene targeting in plants. EMBO J. 7:4021-4026, 1988.
58) Pruitt, R. E. & Meyerowitz, E. M. Characterization of the genome of Arabidopsis thaliana. J Mol Biol 187:169-183, 1986.
59) Puchta, H. et al. Somatic intrachromosomal homologous recombination events in populations of plant siblings. Plant Mol Biol 28:281-292, 1995a.
60) Puchta, H. et al. Induction of intrachromosomal homologous recombination in whole plants. Plant J 7:203-210, 1995b.

61) Puchta, H. Gene replacement by homologous recombination in plants. Plant Mol Biol 48:173-182, 2002.
62) Reiss, B. et al. RecA protein stimulates homologous recombination in plants. Proc Natl Acad Sci USA 93:3094-3098, 1996.
63) Reiss, B. et al. Targeting of a functional Escherichia coli RecA protein to the nucleus of plant cells. Mol Gen Genet 253:695-702, 1997.
64) Reiss, B. et al. RecA stimulates sister chromatid exchange and the fidelity of double-strand break repair, but not gene targeting, in plants transformed by agrobacterium. Proc Natl Acad Sci USA 97:3358-3363, 2000.
65) Richmond, E. & Peterson, C. L. Functional analysis of the DNA-stimulated ATPase domain of yeast SWI2/SNF2. Nucl Acids Res 24:3685-92, 1996.
66) Schaffer, R. et al. The late elongated hypocotyl mutation of Arabidopsis disrupts circadian rhythms and the photoperiodic control of flowering. Cell 93:1219-29, 1998.
67) Shalev, G. et al. Stimulation of homologous recombination in plants by expression of the bacterial resolvase ruvC. Proc Natl Acad Sci USA 96:7398-7402, 1999.
68) Shen, X. et al. A chromatin remodeling complex involved in transcription and DNA processing. Nature 406:541-544, 2000.
69) Shen, X. et al. Modulation of ATP-dependent chromatin-remodeling complexes by inositol polyphosphates. Science 299: 112-114, (2003)

70) Simon, M. et al. The 3′ to 5′ exonuclease activity located in the DNA polymerase δ subunit of Saccharomyces cerevisiae is required for accurate replication. EMBO J. 10: 2165-2170, 1991.
71) Sitney, K. C. et al. DNA polymerase III, a second essential DNA polymerase, is encoded by the S. cerevisiae CDC2 gene. Cell 56:599-605, 1989.
72) Steger, D. J. et al. Regulation of chromatin remodeling by inositol polyphosphates. Science 299:114-116, 2003.
73) Sugino, A. Yeast DNA polymerases and their role at the replication fork. TIBS 20:319-323, 1995.
74) Swoboda, P. et al. Intrachromosomal homologous recombination in whole plants. EMBO J. 13:484-489, 1994.
75) Thykjaer, T. et al. Gene targeting approaches using positive-negative selection and large flanking regions. Plant Mol Biol 35:523-530, 1997.
76) Torres-Ramos, C. A. et al. Requirement of Yeast DNA polymerase δ in post-replicational repair of UV-damaged DNA. J Biol Chem 272:25445-25448, 1997.
77) Travers, A. An engine for nucleosome remodeling. Cell 96:311-314, 1999.
78) Troelstra, C. et al. ERCC6, a member of a subfamily of putative helicases, is involved in Cockayne's syndrome and preferential repair of active genes. Cell 71:939-53, 1992.
79) van Gool, A. J. et al. RAD26, the functional S. cerevisiae homolog of the Cockayne syndrome B gene ERCC6. EMBO J. 13:5361-5369, 1994.
80) Walden, R. et al. Activation tagging: a means of isolating genes implicated as playing a role in plant growth and development. Plant Mol Biol 26:1521-1528, 1994.
81) Weigel, D. et al. Activation tagging in Arabidopsis. Plant Physiol 122:1003-1013, 2000.
82) Wilson, K. et al. A Dissociation insertion causes a semidominant mutation that increases expression of TINY, an Arabidopsis gene related to APETALA2. Plant Cell 8: 659-671, 1996.
83) Wood, M. et al. An ATPase/helicase complex is an essential cofactor for oncogenic transformation by c-Myc. Mol Cell 5:321-330, 2000.
84) Zhu, T. et al. Targeted manipulation of maize genes in vivo using chimeric RNA/DNA oligonucleotides. Proc Natl Acad Sci USA 96:8768-8773, 1999.
85) Zhu, T. et al. Engineering herbicide-resistant maize using chimeric RNA/DNA oligonucleotides. Nat Biotechnol 18:555-558, 2000.

### BRIEF DESCRIPTION OF THE DRAWINGS

FIG. 1 depicts an alignment of an AtIno80 sequence (SEQ ID NO:1) and public sequence, At3g57300 (SEQ ID NO:3), showing a splicing difference (“Query” refers to AtIno80 sequence; “Sbjct” to public database sequence, gi|18410689|ref|NM_{—}115590.11 (AGI:At3g57300)

### SUMMARY OF THE INVENTION

The present invention provides an isolated nucleic acid, in particular DNA, comprising a sequence having 98.5% or more identity with the sequence depicted in SEQ ID NO: 1 (AtIno80). Also provided are vectors and host cells comprising the nucleic acids of the invention, as well as polypeptides encoded by the nucleic acids.

In a further aspect of the invention, a method for inducing homologous recombination in a cell is provided, comprising modulating the expression or properties of one or more gene products selected from the group consisting of AtIno80 (SEQ ID NOs:1 and 2), At3g57300 (SEQ ID NO:3), Rvb1 (At5g22330; SEQ ID Nos: 4 and 5), Rvb21 (At5g67630; SEQ ID NOs: 6 and 7), Rvb22 (At3g49830: SEQ ID NOs: 8 and 9), At3g57290 (SEQ ID NO: 10), AtArp5.1 (At3g12380; SEQ ID NOs: 11 and 12), AtArp5.2 (At5g56180; SEQ ID NOs: 13 and 14), AtArp5.3 (At3g60830; SEQ ID Ns: 15 and 16) and AtArp8 (At5g43500; SEQ ID Nos: 17 and 18), their homologues, fragments or derivatives. In one embodiment, modulation is achieved by increasing expression of the gene product, such as by introducing a nucleic acid encoding the gene product into the cell operably linked to a promoter; and allowing transcription and translation of the gene in an amount sufficient to affect homologous recombination in said cell.

The method can be used to increase somatic homologous recombination and/or meiotic homologous recombination. The promoter can be an inducible promoter, a tissue-specific promoter, a constitutive promoter or a meiosis-specific promoter, depending on the desired effect.

Also provided is a method of increasing gene targeting to a desired locus in a host cell comprising introducing a desired gene into a host cell, modulating the expression or properties of one or more gene products selected from the group consisting of AtIno80, At3g57300, Rvb1, Rvb21, Rvb22, At3g57290, AtArp5.1, AtArp5.2, AtArp5.3 and AtArp8, or functional fragments, derivatives and homologues thereof in the host cell, and detecting integration of the desired gene at a selected locus in the genome of the host cell.

### DETAILED DESCRIPTION OF THE INVENTION

The present inventors have used a direct screening approach to identify mutants of Arabidopsis thaliana showing increased frequencies of somatic recombination, by visualizing recombination events in living plants from a mutagenized population and directly isolating plants with the desired phenotype. The description below describes a genetic screen and an Arabidopsis mutant sm22 derived from it, and the associated plant genes responsible for the altered recombination phenotype.

Existing technologies for gene targeting in plants are very inefficient. The modulation of the expression or properties of one or more gene products selected from the group consisting of AtIno80 (SEQ ID NOs:1 and 2), At3g57300 (SEQ ID NO:3), Rvb1 (At5g22330; SEQ ID Nos: 4 and 5), Rvb2(1 and 2; also referred to herein as Rvb21, At5g67630; SEQ ID NOs: 6 and 7 and Rvb22, At3g49830: SEQ ID NOs: 8 and 9, respectively), At3g57290 (SEQ ID NO: 10), AtArp5.1 (At3g12380; SEQ ID NOs: 11 and 12), AtArp5.2 (At5g56180; SEQ ID NOs: 13 and 14), AtArp5.3 (At3g60830; SEQ ID Ns: 15 and 16) and AtArp8 (At5g43500; SEQ ID Nos: 17 and 18), their homologues (including orthologs), fragments or derivatives increases the efficiency of gene targeting events and facilitates the routine manipulation of the genome of higher plants by homologous recombination. For the purposes of this disclosure, to avoid repetition, reference to the above group of gene products is meant to include reference to each gene individually, i.e., the modulation of the expression or properties of AtIno80, the modulation of the expression or properties of At3g57300, and so on.

An in vivo screen for Arabidopsis mutants has been devised to allow direct detection of mutants with altered recombination. As a result of the screen, mutant plants with a more than 10-fold increased or altered frequency of somatic recombination events are provided, as well as the plant gene AtIno80. One or more of AtIno80, At3g57300, Rvb1, Rvb21, Rvb22, At3g57290, AtArp5.1, AtArp5.2, AtArp5.3 and AtArp8 or orthologs from other plant species are affected in these mutant plants. The screen allows the identification of mutant plants, and plant genes with a strong effect on recombination having little or no undesired side effects on the plant. An increase in homologous recombination frequency is useful to achieve an increased efficiency of gene targeting in plants.

Within the context of the present invention reference to a gene is to be understood as reference to a DNA coding sequence associated with regulatory sequences, which allow transcription of the coding sequence into RNA such as mRNA, rRNA, tRNA, snRNA, sense RNA or antisense RNA. Examples of regulatory sequences are promoter sequences, 5′ and 3′ untranslated sequences, introns, and termination sequences.

A promoter is understood to be a DNA sequence initiating transcription of an associated DNA sequence, and may also include elements that act as regulators of gene expression such as activators, enhancers, or repressors.

Expression of a gene refers to its transcription into RNA or its transcription and subsequent translation into protein within a living cell. In the case of antisense constructs expression refers to the transcription of the antisense DNA only. The term transformation of cells designates the introduction of nucleic acid into a host cell, particularly the stable integration of a DNA molecule into the genome of said cell. Any part or piece of a specific nucleotide or amino acid sequence is referred to as a component sequence or fragment.

In one aspect of the invention, nucleic acids and polypeptides are provided that can modulate homologous recombination. A nucleic acid according to the present invention comprises a sequence having 98.5%, 99%, 99.5% or more identity with the sequences depicted in SEQ ID NO:1. The nucleic acid can be DNA or RNA, such as, mRNA, rRNA, tRNA, snRNA, sense RNA or antisense RNA. Also provided is a vector comprising the nucleic acid of the invention, as well as host cells comprising the vector or nucleic acid of the invention. Suitable vectors and host cells are described in more detail below. Also provided are polypeptides encoded by the nucleic acids of the invention.

In a further aspect of the invention, methods for increasing homologous recombination are provided by modulating the expression or properties of one or more gene products selected from the group consisting of AtIno80, At3g57300, Rvb1, Rvb21, Rvb22, At3g57290, AtArp5.1, AtArp5.2, AtArp5.3 and AtArp8. In order to increase homologous recombination several methods are useful depending on the gene and the gene targeting technique employed. Typically, modulation will mean increasing the activity of the gene product, which can easily be achieved by methods known in the art.

In one embodiment, the desired gene is overexpressed in a host cell in an amount sufficient to increase homologous recombination in the host cell. By “overexpression”, it is meant increasing the amount of desired gene product in a host cell, compared to untreated cells. A simple way to achieve overexpression is to produce transgenic host cells, in particular transgenic plants, carrying a construct (vector) that ectopically overexpresses the sequence of interest under the control of a suitable promoter, such as the 35S CaMV, MAS (mannopine synthase) or ubiquitin promoter.

In another embodiment, an inducible promoter is used to allow an increase in homologous recombination frequency at the time and place needed, for example, for gene targeting.

Alternatively, the construct increasing recombination can be provided at the same time as the targeting construct by co-transformation, the effect is then achieved by the transient expression of the construct containing the said genes.

Functional fragments, homologues (including orthologs) or derivatives can be easily identified by alignment with the sequences referred to above. In general two approaches exist to sequence alignment. Algorithms as proposed by Needleman & Wunsch and by Sellers align the entire length of two sequences providing a global alignment of the sequences. The Smith-Waterman algorithm on the other hand yields local alignments. A local alignment aligns the pair of regions within the sequences that are most similar given the choice of scoring matrix and gap penalties. This allows a database search to focus on the most highly conserved regions of the sequences. It also allows similar domains within sequences to be identified. To speed up alignments using the Smith-Waterman algorithm both BLAST (Basic Local Alignment Search Tool) and FASTA place additional restrictions on the alignments.

Within the context of the present invention alignments are conveniently performed using BLAST, a set of similarity search programs designed to explore all of the available sequence databases regardless of whether the query is protein or DNA. Version BLAST 2.0 (Gapped BLAST) of this search tool has been made publicly available on the internet (currently http://www.ncbi.nfm.nih.gov/BLAST/). It uses a heuristic algorithm which seeks local as opposed to global alignments and is therefore able to detect relationships among sequences which share only isolated regions. The scores assigned in a BLAST search have a well-defined statistical interpretation. Particularly useful within the scope of the present invention are the blastp program allowing for the introduction of gaps in the local sequence alignments and the PSI-BLAST program, both programs comparing an amino acid query sequence against a protein sequence database, as well as a blastp variant program allowing local alignment of two sequences only. Said programs are preferably run with optional parameters set to the default values.

Sequence alignments using BLAST can also take into account whether the substitution of one amino acid for another is likely to conserve the physical and chemical properties necessary to maintain the structure and function of the protein or is more likely to disrupt essential structural and functional features of a protein. Such sequence similarity is quantified in terms of a percentage of “positive” amino acids, as compared to the percentage of identical amino acids and can help assigning a protein to the correct protein family in border-line cases.

Specific examples of DNA and encoded proteins according to the present invention are described in SEQ ID NOS: 1-18. The putative ATPase/helicase AtIno80 may, as demonstrated in yeast, be part of a complex containing one or more of Rvb1, Rvb2, Arp5 and Arp8. All these proteins may be useful in increasing homologous recombination frequency.

Typically, functional fragments or derivatives are characterized by an amino acid sequence comprising a component sequence of at least 150 amino acid residues having 40% or more identity with an aligned component sequence of the one or more of the polypeptides encoded by the DNA of SEQ ID NOs: 1, 3, 4, 6, 8, 10, 11, 13, 15, 16 or 18. Preferably the amino acid sequence identity is higher than 50% or even higher than 55%. Most preferably the protein sequence is that of SEQ ID NO:2.

DNA encoding proteins according to the present invention can be isolated from monocotyledonous and dicotyledonous plants. Preferred sources are corn, sugarbeet, sunflower, winter oilseed rape, soybean, cotton, wheat, rice, potato, broccoli, cauliflower, cabbage, cucumber, sweet corn, daikon, garden beans, lettuce, melon, pepper, squash, tomato, or watermelon. However, they can also be isolated from mammalian sources such as mouse or human tissues. The following general method, can be used, which the person skilled in the art knows to adapt to the specific task. A single stranded fragment of the desired gene consisting of at least 15, preferably 20 to 30 or even more than 100 consecutive nucleotides is used as a probe to screen a DNA library for clones hybridizing to said fragment. The factors to be observed for hybridization are described in Sambrook et al, Molecular cloning: A laboratory manual, Cold Spring Harbor Laboratory Press, chapters 9.47-9.57 and 11.45-11.49, 1989. Hybridizing clones are sequenced and DNA of clones comprising a complete coding region encoding a protein characterized by an amino acid sequence comprising a component sequence of at least 150 amino acid residues having 40% or more sequence identity to the protein sequence encoded by the desired gene is purified. Said DNA can then be further processed by a number of routine recombinant DNA techniques such as restriction enzyme digestion, ligation, or polymerase chain reaction analysis. The disclosure of the nucleotide sequences in SEQ ID NOs: 1, 3, 4, 6, 8, 10, 11, 13, 15, 16 and 18 enables a person skilled in the art to design oligonucleotides for polymerase chain reactions which attempt to amplify DNA fragments from templates comprising a sequence of nucleotides characterized by any continuous sequence of 15 and preferably 20 to 30 or more basepairs of the desired gene.

Suitable vectors for practicing the methods of the invention are well known in the art. Similalry, host cells can be derived from monocotyledonous or dicotyledonous plants. Preferred sources are corn, sugarbeet, sunflower, winter oilseed rape, soybean, cotton, wheat, rice, potato, broccoli, cauliflower, cabbage, cucumber, sweet corn, daikon, garden beans, lettuce, melon, pepper, squash, tomato, or watermelon. However, host cells can also be isolated from other sources, including mammalian sources such as mouse or human cells, in particular stem cells. It is preferred that mammalian homologues are used in mammalian cells.

The methods for increasing homologous recombination are useful to obtain gene targeting so that a gene of interest is introduce into the genome at a desired locus, instead of randomly. For some hosts, in particular crop plants, the gene is preferably expressed in a selected tissue where expression is needed. This is easily achieved by the use of tissue specific promoter. Thus, the present invention provides a method for increasing somatic homologous recombination and increasing gene targeting by modulating the expression or properties of one or more gene products selected from the group consisting of AtIno80, At3g57300, Rvb1, Rvb21, Rvb22, At3g57290, AtArp5.1, AtArp5.2, AtArp5.3 and AtArp8 and fragments, derivatives and homologues thereof, essentially as described above. As is apparent to one of ordinary skill in the art, the corresponding ortholog is preferably used for any particular plant. For example, the corn ortholog of Ino80 is used (or modulated) to increase recombination in corn.

The methods are also useful to improve meiotic recombination, thereby facilitating breeding of species, in which genes encoding a particular phenotype are transferred between plants. Crossing in an interesting trait from another variety or species into a given variety by conventional breeding is a very time and labour-intensive process. Several generations of back-crosses have to be carried out to eliminate the undesired genetic material of the donor species, while maintaining the desired phenotype or trait. Using the methods described above for increasing homologous recombination, meiotic recombination frequencies can be increased, preferably by expressing the desired gene under the control of a meiosis-specific promoter or inducible promoter, the breeding process is speeded up. Thus, the present invention provides a method for increasing meiotic recombination by modulating the expression or properties of one or more gene products selected from the group consisting of AtIno80, At3g57300, Rvb1, Rvb21, Rvb22, At3g57290, AtArp5.1, AtArp5.2, AtArp5.3 and AtArp8 and fragments, derivatives and homologues thereof, essentially as described above.

The Examples below are provided for illustrative purposes and are in no way intended to be limiting to the invention.

### EXAMPLES

### Example 1

### Identification of sm22 Gene Effective in Increasing Homologous Gene Recombination

We have used for our screening a newly constructed a transgenic Arabidopsis thaliana line that carries a recombination reporter construct based on the firefly luciferase gene. The structure of the reporter construct—two segments of the luciferase gene arranged as inverted repeats—is comparable to that of the previously described beta-glucuronidase reporter (Swoboda et al. 1994, Puchta et al. 1995a, Puchta et al. 1995b), but offers the advantage that recombination events can be detected in living plants. Luciferase activity in cells in which recombination has restored an intact luciferase gene can be detected by light emission after application of the substrate D-luciferin using a high-sensitivity CCD camera (Millar et al. 1992, Millar et al. 1995a, Millar et al. 1995b, Michelet & Chua 1996).

To induce hyperrecombination mutations in the luciferase recombination reporter line, we used T-DNA activation tagging with a mutagenic construct (pAC102). “Activation tagging” refers to the transcriptional activation of endogenous plant genes by random integration of a construct that carries promoter or enhancer sequences. One published approach for “activation tagging” is the introduction, via Agrobacterium-mediated gene transfer, of a T-DNA carrying several copies of the cauliflower mosaic virus (CaMV) ³⁵S enhancer (Fang et al. 1989), which can activate the expression of heterologous genes over a distance (Hayashi et al. 1992, Walden et al. 1994, Kakimoto 1996, Kardailsky et al. 1999, Weigel et al. 2000). Another published approach is the introduction of a complete, outward-pointing CaMV ³⁵S promoter on a transposable Ds element (Wilson et al. 1996, Schaffer et al. 1998, Fridborg et al. 1999). The construct “pAC102” used for our experiments is a combination of these previously described elements: it is a binary vector carrying a T-DNA that can be transferred to plants that contains a complete, outward-pointing copy of the CaMV ³⁵S promoter/enhancer close to the right T-DNA border. Thus, this construct combines the ease of application of T-DNA gene transfer with the genetic ability of a complete promoter, avoiding some of the drawbacks of enhancer-only constructs (Weigel et al. 2000).

In principle, the activation tagging construct can cause several kinds of mutations after integration in the plant genome: gene disruption by insertion within a coding sequence, activation of plant gene expression by action of the CaMV 35S enhancer, direct expression of a plant gene from the CaMV 35S promoter on the T-DNA, or down-regulation of expression by antisense RNA production driven from the CaMV ³⁵S promoter. The pAC102 T-DNA carries in addition to the ³⁵S promoter a complete copy of the pUC cloning vector to facilitate gene cloning by plasmid rescue (Dilkes & Feldmann 1998), and a sulfonamide resistance marker (Guerineau et al. 1990, Reiss et al. 1996) for selection of transgenic plants.

We transformed 13.000 three-week old Arabidopsis ecotype Columbia plants from the luciferase recombination reporter line with the activation-tagging T-DNA construct “pAC102” by Agrobacterium-mediated gene-transfer, using the established “floral dip” procedure (Clough & Bent 1998) with a modified infiltration buffer, in which the Silwet L-77 detergent was replaced by 0.05% Extravon® (Ciba). Seeds from the infiltrated plants were harvested three weeks after infiltration. Transgenic progeny carrying the pAC102 activation tagging T-DNA were selected by sowing seeds on perlite substrate drenched with Gamborg B5 mineral medium (Gamborg et al. 1968) containing 10 mg/l sulfadiazine (Sigma), and transferring surviving individuals after 10 days to soil. About 20,000 sulfonamide-resistant plants were isolated; they represent independent transformants with the pAC102 T-DNA activation tagging construct integrated at different random positions in the Arabidopsis genome.

When individual plants had grown to the 10-leaf stage, they were assayed for luciferase activity to detect somatic recombination events. Batches of 25 plants were sprayed with the substrate D-luciferin and pictures (typically two) were taken with a “Astrocam” (Gloor Instruments, Uster) by integrating photons over 15 min. Background noise and cosmic radiation was filtered out by correlating both images using the minimum function. Plants showing an increased number of sectors with luciferase activity relative to the average of the population were observed with a frequency of about 1 in 500 plants.

As a result of the screen, a hyperrecombination mutant plant was isolated called sm22. The original hyper-recombination phenotype of sm22 plant shows an enhancement of about 20- to 50-fold for homologous recombination in the reporter line. No other obvious phenotype was seen and the seed yield was normal. Sulfonamide selection in the second generation (T2) revealed a 2/1 or 3/1 segregation of resistant seedlings, thus showing that there is only 1 locus (or 2 closely related loci) with an active T-DNA inserted. However, the T2 recombination phenotype was even lower (less or same number of recombination events per plant) than in the wild type.

After HindIII digestion of T1 callus genomic DNA prepared essentially according to the method of the Nucleon Phytopure protocol and Plant DNA extraction kit (Amersham), plasmid rescue was applied (Dilkes & Feldmann 1998, Mathur et al. 1998), which gave rise to two independent junction fragments. Briefly, we digested plant genomic DNA with HinDIII, circularized the resulting fragments by ligation at low DNA concentration, and transformed the ligation mixture into competent E. coli TOP10 cells (commercially available from INVITROGEN) by electroporation. Since the HindIII fragments containing the fusion joint between plant DNA and the right end of the activation tagging construct carry a plasmid origin and the ampicillin resistance gene (bla) contributed by pAC102, circularization of such fragments will result in a functional bacterial plasmid and confer ampicillin resistance to the E. coli cells.

Several colonies were obtained after plating the transformed bacteria on selection medium containing ampicillin. Plasmid DNA of these transformants was prepared and characterized by restriction analysis. To determine the nature of the plant sequences joined to the right end of the T-DNA, the plant DNA insert from these rescued plasmids was sequenced from both sides, using one custom sequencing primer complementary to the T-DNA right end reading towards the plant DNA, and the standard M13 reverse sequencing primer, reading from the pAC102 vector sequences into the plant DNA insert from the other end. The obtained DNA sequences were compared to the GenBank nucleotide database using the BLASTN search program.

Two insertions were identified. The first one corresponds to a single T-DNA insertion without deletion (left border, LB, junction sequenced) in the N-terminal region of a putative ATPase/helicase gene, in antisense orientation. The second T-DNA inserted in a gene with no obvious relationship to homologous recombination (gb AF082176_{—}1) and does not confer sulfonamide resistance. Six (T3) resistant families were analysed by PCR and Southern. Only one family contained some plants with the second insertion whereas all families have the helicase insertion site.

In subsequent generations, homozygous plants for the helicase insertion site were obtained. The homologous recombination frequency of heterozygous and homozygous plants for this insertion site was at least 50% and 15%, respectively, and up to 80% and 20%, respectively, of the wild type level.

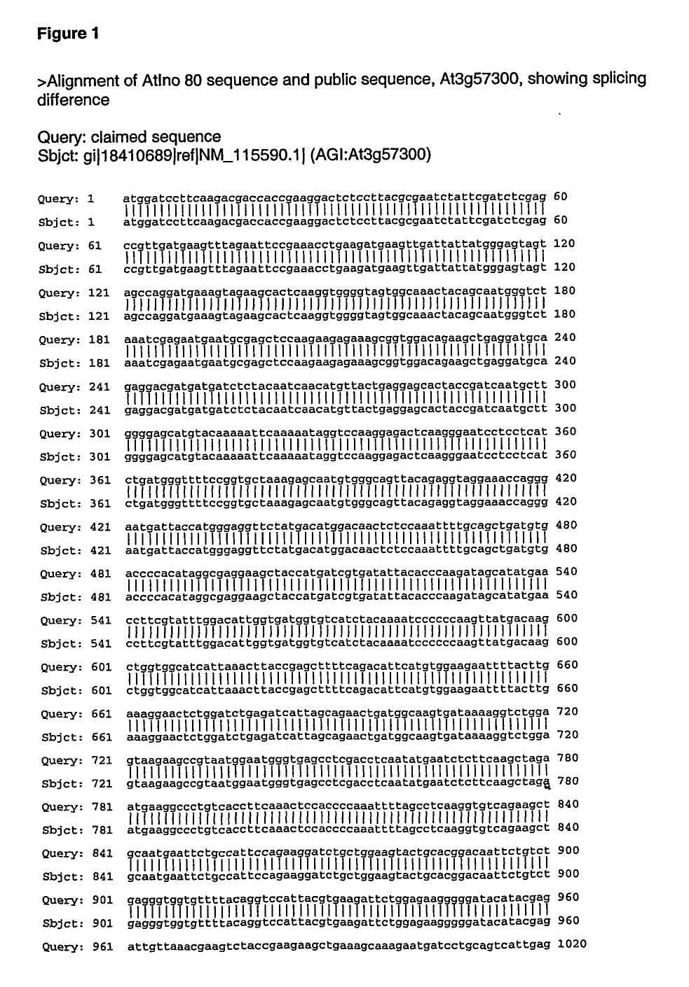

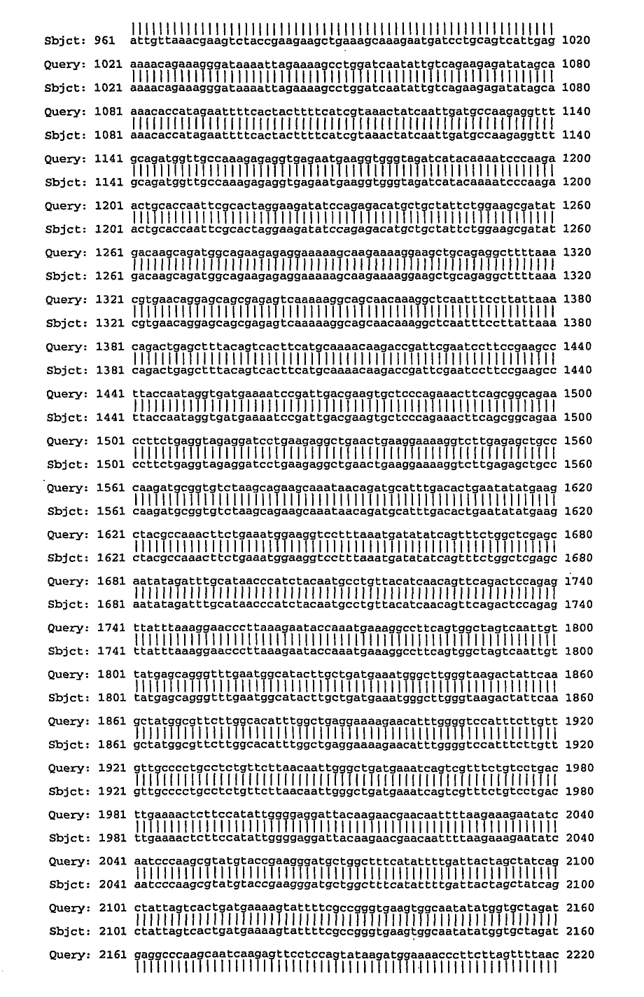

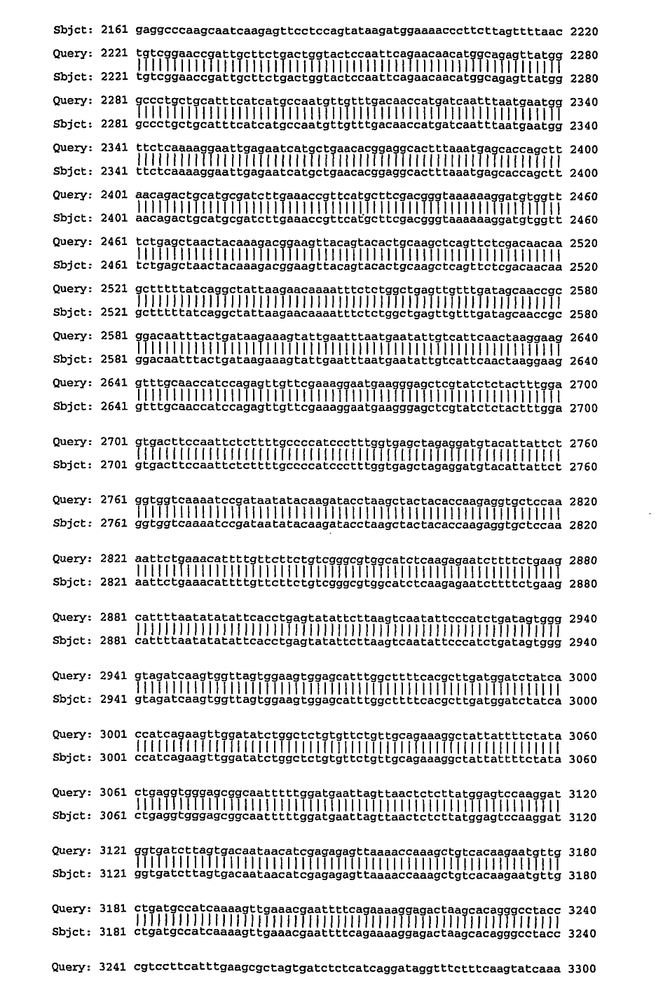

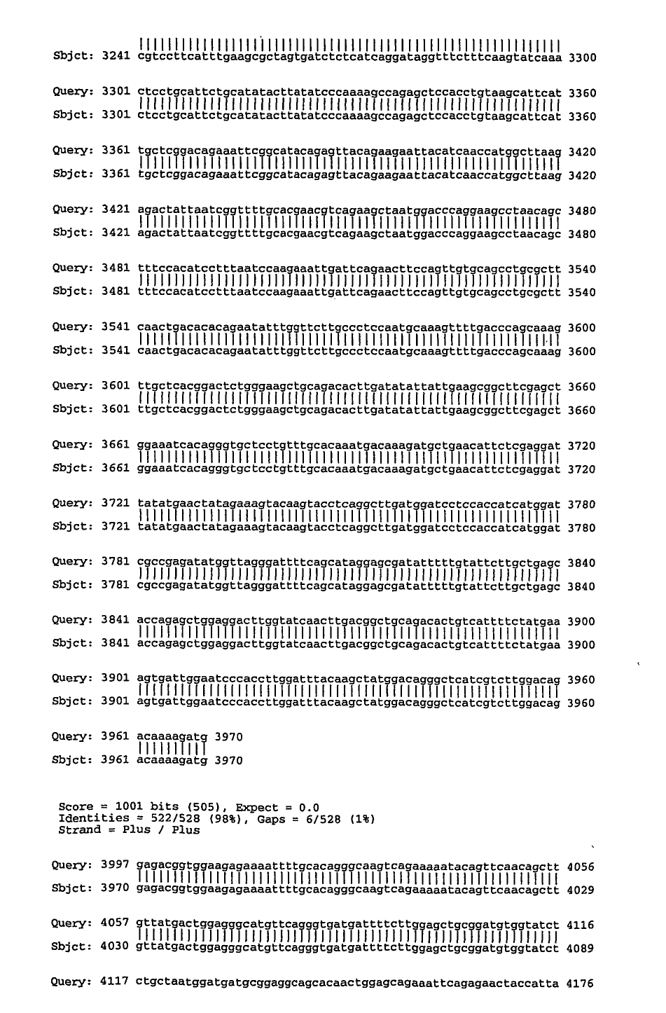

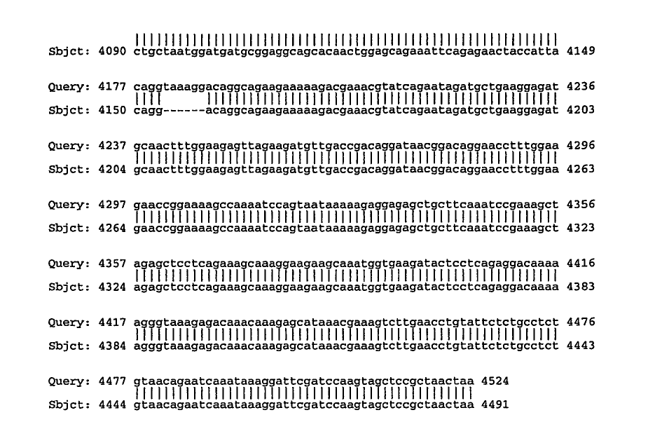

The complete cDNA (4.8 kb) was cloned in two steps. First, a public EST containing the 3′ part was sequenced. Then the 5′ part of the cDNA was amplified by RT-PCR on Col-0 (Arabidopsis Columbia ecotype, wild type) callus RNA (prepared with the Qiagen RNAeasy Plant Kit), using primers in the 5′ untranslated region including a stop codon in frame with the predicted ATG (sm5UT) to make sure that the complete 5′ part of the cDNA was amplified. The primer sequences were sm5UT: ctagaagcttttaaggatTAAgactctcc (SEQ ID NO:19) and for 3′ primer: ctcgtatgtatcccccttctcc (SEQ ID NO:20). The coding sequence is provided as SEQ ID NO.1 and is similar to but not identical to At3g57300 (see FIG. 1).

The predicted helicase gene (8 kb genomic DNA) has about 23 exons encoding a protein of 1507 amino acids (SEQ ID NO:2). It is predicted to be an ATPase of the Swi2/Snf2 family, and contains several nuclear localization signals (NLS). The ATPase/helicase gene is the putative Arabidopsis ortholog of the yeast Ino80pNGL150c protein (Ebbert et al. (1999), Shen et al. (2000)). Homologs exist in yeast, budding yeast, Drosophila and human. These four homologues have several highly conserved regions including the six motifs of the SWI2/SNF2 helicase domain. Several NLS suggest a nuclear localization of the gene product.

In the sm22 heterozygous and homozygous mutant, the level of Ino80 transcript is respectively about 50% of the wild type situation or absent, as measured by semi-quantitative RT-PCR (5′ At3g57300 primer: TGATGGATCTATCACCATCAG, SEQ ID NO:21; 3′ At3g57300 primer: ggtgggattccaatcactttc, SEQ ID NO:22) and by northern blot hybridization. For this, the RNA was extracted from 2 weeks old seedlings using the QIAGEN RNAeasy plant extraction kit following the manufacturer's instructions. Together with the decrease of homologous recombination in sm22 plants described above (50% in heterozygous plants, 15% in homozygous mutant), this result shows that the level of Ino80 gene product might positively and directly regulate the homologous recombination frequency, making this gene a choice candidate to positively regulate homologous recombination.

Results with a recombination reporter line 1445 (Gherbi et al. 2001) overexpressing the INO80 cDNA under the control of the ³⁵S promoter and with an N-terminal HA-tag interrupted by an intron show upregulation of homologous recombination providing evidence that INO80 upregulates homologous recombination.

The yeast homolog (Ebbert et al., 1999), INO80(=YGL150c) is part of a big complex >1 MDa (monomeric form is 171 KDa), containing two essential helicases Rvb1p and Rvb2p and actin related proteins (arp) Arp4, Arp5 and Arp8 (Cho et al. 2001; Jonsson et al. 2001; Wood et al. 2000). Thus, the involvement of Ino80 in homologous recombination implicates the activity of these other genes in homologous recombination. In eukaryotes Human Rvb1p and Rvb2p are also known (Kanemaki 1999, Ikura et al. 2000, Shen et al. 2000).

In Arabidopsis thaliana we found three genes closely related to Rvbs from other organisms, AtRvb1 (SEQ ID NO:4, SEQ ID NO:5), AtRvb21 (SEQ ID NO: 6, SEQ ID NO:7) and AtRvb22 (SEQ ID NO:8, SEQ ID NO:9). The three genes are expressed (RT-PCR) and some of them are positively regulated by genotoxic stress (UVc, bleomycin). For treatment with Bleomycin (BLM) 2 week-old Arabidopsis seedlings were placed under sterile conditions in liquid GM medium containing 10⁻⁶M of BLM (Sigma) or 100 ppm of MMS (Fluka, Switzerland). For UV-C irradiation (6000 ergs) 2 week-old seedlings were irradiated with light provided by a HNS 55W OFR lamp (Osram). After treatment, plants were harvested at several time points (30 min, 1 h, 4 h and 12 h) and RNA extracted as described above. Then semi-quantitative RT-PCR analysis was performed with the following primers AtIno80 (TGATGGATCTATCACCATCAG, SEQ ID NO:23; ggtgggattccaatcactttc, SEQ ID NO:24) AtRvb1 (tttgatgggccaaatgatg, SEQ ID NO:25; cttccaaCCTAGGtgagatgtttcaacaaaatgtgc, SEQ ID NO:26) AtRvb21 (tcaacagcaggacacaagg, SEQ ID NO:27; cccaatgCCTAGGaaatcegagttcaacatcctaatc, SEQ ID NO:28) AtRvb22 (acaaaccagatatcagcacatgg, SEQ ID NO:29; aacaagtactcgctctcatgctc, SEQ ID NO:30).

To characterize further the AtIno80-1 HR deficiency, we subjected the mutant to various genotoxic stresses. In parallel with the original ino80 mutant we also tested two allelic mutants of INO80 from the publicly available SAIL mutant collection. Neither bleomycin nor Mitomycin-C or UV-C sensitivity was shifted in the Atino80-1 mutants, in any of the various conditions tested. All the Atino80-1 alleles seem to be slightly hypersensitive to MMS (methyl methanesulfonate), which is also known to induce DNA double-strand breaks. The difference in sensitivity is visible at 60 and 80 ppm of MMS on root elongation and rosette growth. Most mutations affecting DNA repair or recombination also give rise to changes in the sensitivity to DNA damaging agents. We challenged Atino80-1 mutant plants with various treatments known to induce DNA damage and recombination. None of the tested agents (UV-C, bleomycin, Mitomycin-C and MMS) gave rise to a shift in sensitivity, with the exception of a slight change for MMS. This suggests a difference for the role of INO80 in plants compared to yeast (Ebbert et al., 1999; Shen et al. 2000) and supports the use of AtlNO80 to regulate homologous recombination without affecting the major repair pathway in plants.

In the sm22 background the steady state level of AtRvb21 and AtRvb22 was shown to be down-regulated using RT-PCR on RNA extracted as above mentioned. This indicates that the components of the putative Arabidopsis INO80 complex show co-regulation at the transcriptional level, supporting the use of Arabidopsis Rvb1, Rvb21 and Rvb22 and the Arabidopsis Arp protein orthologs to manipulate homologous recombination frequency in plants.

### Example 2

### AtRvb1 as Positive Regulator of Homologous Recombination

As describe above (Example 1), the original recombination-up phenotype found in sm22 can be associated with an effect mediated by the Arabidopsis Rvb1 and 2 orthologs. Thus, AtRvb1 can be used as a positive regulator of homologous recombination.

### Example 3

### AtRvb21 as Positive Regulator of Homologous Recombination

As describe above (Example 1), the original recombination-up phenotype found in sm22 can be associated with an effect mediated by the Arabidopsis Rvb1 and 2 orthologs. Thus, AtRvb21 can be used as a positive regulator of homologous recombination.

### Example 4

### AtRvb22 as Positive Regulator of Homologous Recombination

As describe above (Example 1), the original recombination-up phenotype found in sm22 can be associated with an effect mediated by the Arabidopsis Rvb1 and 2 orthologs. Thus, AtRvb22 can be used as a positive regulator of homologous recombination.

### Example 5

### At3g57290 as Positive Regulator of Homologous Recombination

In the sm22 mutant (Example 5), the At3g57290p gene is potentially overexpressed by the 35S Enhancer/promoter. Over expression of this gene in the sm22 context or directly with a 35S promoter can be carried out to reproduce the original recombination-up phenotype. The phenotype was lost in the second generation (Example 1), at which point At3g57290 is not overexpressed any longer allowing a temporal ability to modulate homologous recombination.

### Example 6

### AtArp as Positive Regulators of Homologous Recombination

As describe above (Example 1), the original recombination-up phenotype found in sm22 can be associated with an effect mediated by other components of the Arabidopsis INO80 complex, such as the Arp homolog AtArp5.1, AtArp5.2, AtArp5.3 and/or AtArp8. Any of these Arp hmologues can be used as a positive regulator of homologous recombination, alone or in combination.

All publications referred to herein as well as the disclosure of GB patent application 0214896.3 are incorporated by reference as if each is referred to individually.

1. An isolated nucleic acid comprising a sequence having 98.5% or more identity with the sequence depicted in SEQ ID NO:1.

2. The nucleic acid of claim 1, wherein said nucleic acid is DNA.

3. A vector comprising the nucleic acid if claim 2.

4. A host cell comprising the vector or nucleic acid of claim 3.

5. A polypeptide encoded by the isolated nucleic acid of claim 1.

6. A method for inducing homologous recombination in a cell, said method comprising modulating the expression or properties of one or more gene products selected from the group consisting of AtIno80, At3g57300, Rvb1, Rvb21, Rvb22, Arp5 and Arp8, fragments, derivatives and homologues thereof,.

7. The method of claim 11, said method comprising increasing expression of said gene product.

8. The method of claim 12, said method comprising introducing a nucleic acid encoding said gene product into said cell operably linked to a promoter; and allowing transcription and translation of said gene in an amount sufficient to affect homologous recombination in said cell.

9. The method of claim 13, wherein said homologous recombination is somatic homologous recombination.

10. The method of claim 13, wherein said homologous recombination is meiotic homologous recombination.

11. The method of claim 13, wherein said promoter is an inducible promoter.

12. The method of claim 13, wherein said promoter is a tissue-specific promoter.

13. The method of claim 13, wherein said promoter is a constitutive promoter.

14. The method of claim 13, wherein said promoter is a meiosis-specific promoter.

15. A method of increasing gene targeting to a desired locus in a host cell, said method comprising introducing a desired gene into a host cell, modulating the expression or properties of one or more gene products selected from the group consisting of AtIno80, At3g57300, Rvb1, Rvb21, Rvb22, Arp5 and Arp8 or functional fragments, derivatives and homologues thereof in said host cell, and detecting integration of said desired gene at a selected locus in the genome of said host cell.
